(19) United States
(12) Patent Application Publication
(10) Pub. No.: US 2025/0263461 A1
(43) Pub. Date: Aug. 21, 2025

(54) RECOMBINANT T-CELL RECEPTORS THAT BIND THE NY-ESO-1 AND/OR LAGE-1A CANCER ANTIGENS

(71) Applicant: T-Cure Bioscience, Inc., Sherman Oaks, CA (US)

(72) Inventors: Ying Q. Lin, Northridge, CA (US); Nicholas S. Davis, North Hollywood, CA (US); Maize Wang, Oak Park, CA (US); Gang Zeng, Calabasas, CA (US); Erika Von Euw, Northridge, CA (US)

(21) Appl. No.: 18/250,347

(22) PCT Filed: Jun. 18, 2021
(86) PCT No.: PCT/US2021/038086

§ 371 Date: Apr. 24, 2023

Related U.S. Application Data
(60) Provisional application No. 63/106,329, filed on Oct. 27, 2020.

(51) Int. Cl. C07K 14/725; A61K 40/11; A61K 40/32; A61K 40/42; A61P 35/00; C12N 5/0783

(52) U.S. Cl. CPC C07K 14/7051; A61K 40/11; A61K 40/32; A61K 40/4267; A61K 40/4269; A61P 35/00; C12N 5/0636; C12N 2510/00

(57) ABSTRACT

The present invention relates to recombinant T-cell receptors that bind specifically, in a MHC restricted manner, to a particular epitope present in the shared cancer-testis antigen known as NY-ESO-1 and/or a particular epitope present in the closely related antigen LAGE-1. The invention provides T-cell receptor related polypeptides, fragments, and functional variants thereof, as well as nucleic acids encoding the T-cell receptor polypeptides of the invention, recombinant expression vectors, and genetically modified cells (for example, T-cells) expressing the T-cell receptors, and their use in methods for diagnosing, treating or preventing cancer in a subject.

### CROSS REFERENCE TO RELATED APPLICATIONS

This application is a U.S. national stage application, filed under 35 U.S.C. § 371, of International Application No. PCT/US2021/038086, filed on Jun. 18, 2021, which claims priority to and the benefit of U.S. Provisional Application No. 63/106,329, filed on Oct. 27, 2020, the entire disclosure of each of which is incorporated by reference herein in its entirety for all purposes.

### SEQUENCE LISTING

The instant application contains a Sequence Listing which has been submitted electronically in ASCII format and is hereby incorporated by reference in its entirety. Said ASCII copy, created on Sep. 25, 2023, is named TCB-002WOUS_SL.txt and is 197,232 bytes in size.

### FIELD OF THE INVENTION

The invention relates generally to T-cell receptors and their use in therapy, and more specifically relates to T-cell receptors that bind, in an MHC restricted manner, to the shared cancer-testis antigen known as NY-ESO-1 and/or the closely related antigen known as LAGE-1a.

### BACKGROUND OF THE INVENTION

T-cells have been found to play an important role in controlling the growth and proliferation of cancer cells and tumors. CD8⁺ T-cells appear to play a role in directly targeting cancer cells, whereas tumor antigen-specific CD4⁺ helper T-cells appear to play a critical role in the initiation, proliferation, maintenance, and co-ordination of overall anti-tumor immune responses, including CD8⁺ T-cell and antibody mediated immune responses. CD8⁺ T-cell-based immunotherapies in particular have shown encouraging results in clinical trials targeting various solid tumors (Robbins et al. (2011) J. CLIN. ONCOL. 29(7): 917; Robbins et al. (2015) CLIN. CANCER RES. 21(5): 1019-27; Rapoport et al. (2015) NAT. MED. 21(8): 914-21).

NY-ESO-1 is a cancer/testis antigen that has been detected in many tumor types, including melanoma, breast cancer, lung cancer, and others, but not in normal tissue except the immune privileged testis (Chen et al. (1997) PROC. NATL. ACAD. SCI. USA 94:1914-1918; Zeng et al. (2000) J. IMMUNOL. 165: 1153-1159). Reactivity of T-cells to the NY-ESO-1 antigen has been demonstrated, in some instances, to be restricted in an HLA-A2 restricted manner (Jager et al. (1998) J. EXP. MED. 187: 265; Wang et al. (1998) J. IMMUNOL. 161(7): 3596-3606). CD8+ T cells expressing TCRs recognizing HLA-A2-restricted NY-ESO-1 and/or LAGE-1a epitopes have led to clinical responses in subjects with melanoma, multiple myeloma and various sarcomas (Robbins et al. (2011) supra; Robbins et al. (2015) supra; Rapoport et al. (2015) supra). Given the limited availability of such immunoreagents, there is an ongoing and unmet need to provide additional new compositions and methods that can be used to for treatment of cancer subjects.

### SUMMARY OF THE INVENTION

The present invention provides a T-cell receptor (TCR), as well as functional fragments or variants thereof, that binds to the core SLLMWITQC (SEQ ID NO: 1) epitope and/or the core SLLMWITQCFL (SEQ ID NO: 28) epitope present in the shared cancer-testis antigen NY-ESO-1 and/or the antigen LAGE-1a in an HLA-restricted manner. For example, the NY-ESO-1 and/or LAGE-1a antigens may be recognized by a T-cell receptor described herein in an HLA-A2 restricted manner. For example, the immunoreactivity to the NY-ESO-1 and/or LAGE-1a antigens can be HLA-A*0201 or HLA-A*0202 restricted.

T-cell receptors comprise two chains referred to as the α- and β-chains, that form a pair on the surface of a T-cell to form a heterodimeric receptor. The T-cell receptor is involved in recognition of MHC-restricted antigens. Each of α- and β-chain comprises two regions, a constant region and a variable region. Each variable region of the α- and β-chains defines three loops, referred to as complementary determining regions (CDRs) known as CDR₁, CDR₂, and CDR₃ that confer the T-cell receptor with antigen binding activity and binding specificity.

In one aspect, the invention provides an isolated, recombinant α-chain of a T-cell receptor immunoreactive with an epitope of a NY-ESO-1 and/or LAGE-1a protein comprising the core amino acid sequence SLLMWITQC (SEQ ID NO: 1) and/or SLLMWITQCFL (SEQ ID NO: 28). The α-chain comprises one or more of the following amino acid sequences: (i) an amino acid sequence of SEQ ID NO: 2 or SEQ ID NO: 87, or an amino acid sequence having greater than 97% identity to the amino acid sequence of SEQ ID NO: 2 or SEQ ID NO: 87; (ii) an α-chain variable region amino acid sequence of SEQ ID NO: 3 or SEQ ID NO: 83, or an amino acid sequence having greater than 96% identity to the amino acid sequence of SEQ ID NO: 3 or SEQ ID NO: 83; (iii) an α-chain CDR₃ amino acid sequence of SEQ ID NO: 7; (iv) an α-chain CDR₁ amino acid sequence of SEQ ID NO: 5; and (v) an α-chain CDR₂ amino acid sequence of SEQ ID NO: 6.

In another aspect, the invention provides an isolated recombinant β-chain of a T-cell receptor immunoreactive with an epitope of a NY-ESO-1 and/or LAGE-1a protein comprising the core amino acid sequence SLLMWITQC (SEQ ID NO: 1) and/or SLLMWITQCFL (SEQ ID NO: 28). The β-chain comprises one or more of the following amino acid sequences: (i) an amino acid sequence of SEQ ID NO: 95 or SEQ ID NO: 99, or an amino acid sequence having greater than 97% identity to the amino acid sequence of SEQ ID NO: 95 or SEQ ID NO: 99; (ii) a β-chain variable region amino acid sequence of SEQ ID NO: 9 or SEQ ID NO: 85, or an amino acid sequence having greater than 97% identity to the amino acid sequence of SEQ ID NO: 9 or SEQ ID NO: 85; (iii) a β-chain CDR₃ amino acid sequence of SEQ ID NO: 13; (iv) a β-chain CDR₁ amino acid sequence of SEQ ID NO: 11; and (v) a β-chain CDR₂ amino acid sequence of SEQ ID NO: 12.

In another aspect, the invention provides a recombinant T-cell receptor immunoreactive with a SLLMWITQC (SEQ ID NO: 1) and/or SLLMWITQCFL (SEQ ID NO: 28) epitope comprising at least one of the foregoing α-chains and at least one of the foregoing β-chains.

In another aspect, the invention provides an isolated, recombinant T-cell receptor immunoreactive with an epitope of a NY-ESO-1 and/or LAGE-1a protein comprising the core amino acid sequence SLLMWITQC (SEQ ID NO: 1) and/or SLLMWITQCFL (SEQ ID NO: 28). The T-cell receptor comprises an α-chain and a β-chain, the α and β chains each comprising a CDR1, CDR2, and a CDR3, wherein the α-chain CDR3 comprises the amino acid sequence of SEQ ID NO: 7, and the β-chain CDR3 comprises the amino acid sequence of SEQ ID NO: 13. Optionally, or in addition, the T-cell receptor α-chain CDR1 comprises the amino acid sequence of SEQ ID NO: 5, and the β-chain CDR1 comprises the amino acid sequence of SEQ ID NO: 11. Optionally, or in addition, the T-cell receptor α-chain CDR2 comprises the amino acid sequence of SEQ ID NO: 6, and the β-chain CDR2 comprises the amino acid sequence of SEQ ID NO: 12.

In another aspect, the invention provides an isolated, recombinant T-cell receptor immunoreactive with an epitope of a NY-ESO-1 and/or LAGE-1a protein comprising the core amino acid sequence SLLMWITQC (SEQ ID NO: 1) and/or SLLMWITQCFL (SEQ ID NO: 28). The T-cell receptor comprises an α-chain variable region comprising an amino acid sequence of SEQ ID NO: 3 or SEQ ID NO: 83, or an amino acid sequence having greater than 96% identity to the amino acid sequence of SEQ ID NO: 3 or SEQ ID NO: 83; and a β-chain variable region comprising an amino acid sequence of SEQ ID NO: 9 or SEQ ID NO: 85, or an amino acid sequence having greater than 97% identity to the amino acid sequence of SEQ ID NO: 9 or SEQ ID NO: 85. Optionally, or in addition, the T-cell receptor α-chain comprises the amino acid sequence of SEQ ID NO: 2 or SEQ ID NO: 87, or an amino acid sequence having greater than 97% identity to the amino acid sequence of SEQ ID NO: 2 or SEQ ID NO: 87, and/or the β-chain comprises the amino acid sequence of SEQ ID NO: 95 or SEQ ID NO: 99, or an amino acid sequence having greater than 97% identity to the amino acid sequence of SEQ ID NO: 95 or SEQ ID NO: 99.

In each of the foregoing aspects, the T-cell receptor is optionally a single chain T-cell receptor, optionally where the α-chain is linked to the β-chain via an amino acid linker. For example, in certain embodiments, the isolated T-cell receptor can comprise the amino acid sequence of SEQ ID NO: 14, which can be encoded by the polynucleotide sequence of SEQ ID NO: 27 or the amino acid sequence of SEQ ID NO: 29, which can be encoded by the polynucleotide sequence of SEQ ID NO: 30.

In certain embodiments of each the foregoing aspects, the T-cell receptor is immunoreactive with the epitope SLLMWITQC (SEQ ID NO: 1) and/or SLLMWITQCFL (SEQ ID NO: 28) in an HLA-A2 restricted manner. For example, the immunoreactivity to the epitope SLLMWITQC (SEQ ID NO: 1) and/or SLLMWITQCFL (SEQ ID NO: 28) can be HLA-A*0201 or HLA-A*0202 restricted.

It is contemplated that, for each of the amino acid sequences provided herein, the sequences optionally include at least one amino acid not present at a given position in T-cell receptor cloned and sequenced in Examples 1 and 2.

It is understood that a T-cell receptor described herein may be conjugated with another binding moiety to produce a bispecific T-cell receptor protein. For example, a T-cell receptor described herein can be associated, for example, covalently or non-covalently associated, to an antibody or an antigen binding fragment thereof to provide a bispecific molecule where the T-cell receptor binds to the SLLMWITQC (SEQ ID NO: 1) and/or SLLMWITQCFL (SEQ ID NO: 28) epitope and the other binding moiety binds to a different antigen. In certain embodiments, the antibody or the antigen binding fragment thereof is capable of modulating an immune response in a subject. In a specific embodiment, the antibody or the antigen binding fragment thereof may be anti-CD3 specific.

In certain embodiments, a T-cell receptor described herein, or a functional fragment thereof, further comprises a detectable label. As a result, the resulting conjugate can be used as a diagnostic or prognostic reagent. Furthermore, in certain embodiments, a T-cell receptor described herein may be associated with a therapeutic agent.

In certain embodiments, a T-cell receptor described herein binds to a SLLMWITQC (SEQ ID NO: 1) peptide/MHC complex with a K_{D} of 500 nM or lower, 400 nM or lower, 300 nM or lower, 200 nM or lower, 175 nM or lower, 150 nM or lower, 125 nM or lower, 100 nM or lower, 75 nM or lower, 50 nM or lower, 25 nM or lower, or 10 nM or lower, as measured by surface plasmon resonance.

In another aspect, the invention provides an isolated, recombinant nucleic acid encoding an α-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). The nucleic acid comprises one or more of the following nucleotide sequences: (i) a nucleotide sequence encoding an amino acid sequence of SEQ ID NO: 2 or SEQ ID NO: 87, or an amino acid sequence having greater than 97% identity to the amino acid sequence of SEQ ID NO: 2 or SEQ ID NO: 87; (ii) a nucleotide sequence encoding an α-chain variable region amino acid sequence of SEQ ID NO: 3 or SEQ ID NO: 83, or an amino acid sequence having greater than 96% identity to the amino acid sequence of SEQ ID NO: 3 or SEQ ID NO: 83; (iii) a nucleotide sequence of SEQ ID NO: 100; (iv) a nucleotide sequence encoding an α-chain CDR₃ amino acid sequence of SEQ ID NO: 7; (iv) a nucleotide sequence encoding an α-chain CDR₁ amino acid sequence of SEQ ID NO: 5; and (vi) a nucleotide sequence encoding an α-chain CDR₂ amino acid sequence of SEQ ID NO: 6.

In another aspect, the invention provides an isolated, recombinant nucleic acid encoding a T-cell receptor β-chain immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). The nucleic acid comprises one or more of the following nucleotide sequences: (i) a nucleotide sequence encoding an amino acid sequence of SEQ ID NO: 95 or SEQ ID NO: 99, or an amino acid sequence having greater than 97% identity to the amino acid sequence of SEQ ID NO: 95 or SEQ ID NO: 99; (ii) a nucleotide sequence encoding a β-chain variable region amino acid sequence of SEQ ID NO: 9 or SEQ ID NO: 85, or an amino acid sequence having greater than 97% identity to the amino acid sequence of SEQ ID NO: 9 or SEQ ID NO: 85; (iii) a nucleotide sequence of SEQ ID NO: 102; (iv) a nucleotide sequence encoding a β-chain CDR₃ amino acid sequence of SEQ ID NO: 13; (v) a nucleotide sequence encoding a β-chain CDR₁ amino acid sequence of SEQ ID NO: 11; and (vi) a nucleotide sequence encoding a β-chain CDR₂ amino acid sequence of SEQ ID NO: 12.

In another aspect, the invention provides an isolated, recombinant nucleic acid encoding a T-cell receptor immunoreactive with an epitope of an NY-ESO-1 and/or LAGE-1a protein comprising the amino acid sequence SLLMWITQC (SEQ ID NO: 1) and/or SLLMWITQCFL (SEQ ID NO: 28). The T-cell receptor comprises an α-chain and a β-chain each comprising a CDR₁, CDR₂, and a CDR₃, wherein the α-chain CDR₃ comprises the amino acid sequence of SEQ ID NO: 7, and the β-chain CDR₃ comprises the amino acid sequence of SEQ ID NO: 13. Optionally, or in addition, the α-chain CDR₁ comprises the amino acid sequence of SEQ ID NO: 5, and the β-chain CDR₁ comprises the amino acid sequence of SEQ ID NO: 11. Optionally, or in addition, the α-chain CDR₂ comprises the amino acid sequence of SEQ ID NO: 6, and the β-chain CDR₂ comprises the amino acid sequence of SEQ ID NO: 12.

It is contemplated that, for each of the nucleic acids described herein, the nucleic acids may encode at least one amino acid not present at a given position in T-cell receptor cloned and sequenced in Examples 1 and 2, and/or the codon usage of the nucleic acids may be optimized to enhance the expression of the α-chain and/or the β-chain of the T-cell receptor in a given subject.

For each of the foregoing nucleic acids, the nucleic acid optionally encodes a single chain T-cell receptor, optionally where the α-chain is linked to the β-chain via an amino acid linker.

In an additional aspect, the invention provides an expression vector, for example, a viral expression vector, comprising one or more of the foregoing nucleic acid sequences. In certain embodiments, the viral vector is a lentivirus vector.

In an additional aspect, the invention provides a genetically modified cell that comprises one or more of the following: (i) an α-chain of a T-cell receptor protein described herein; (ii) a β-chain of a T-cell receptor protein described herein; (iii) a T-cell receptor described herein; (iii) a bispecific T-cell receptor described herein; (iv) a nucleic acid or recombinant expression vector described herein, wherein the cell expresses a T-cell receptor immunoreactive with an epitope comprising the amino acid sequence SLLMWITQC (SEQ ID NO: 1) and/or SLLMWITQCFL (SEQ ID NO: 28).

In certain embodiments of each of the foregoing cells, the cell is an immune-based cell, for example, a CD4⁺ helper T-cell or a CD8⁺ T-cell, or a progenitor cell, for example, a hematopoietic stem cell or a pluripotent stem cell. In certain embodiments, the cell is an autologous cell or a heterologous cell.

In another aspect, the invention provides a method for producing a T-cell immunoreactive with an epitope of an NY-ESO-1 and/or LAGE-1a protein comprising the amino acid sequence SLLMWITQC (SEQ ID NO: 1) and/or SLLMWITQCFL (SEQ ID NO: 28). The method comprises introducing one or more of the foregoing nucleic acids and/or expression vectors into the T-cell.

In certain embodiments of each of the foregoing methods, the T-cell may be a CD4⁺ helper T-cell or a CD8⁺ T-cell. The T-cell may be an autologous cell or a heterologous cell.

In another aspect, the invention provides a pharmaceutical composition comprising a genetically engineered cell produced by the methodologies described herein.

In another aspect, the invention provides a method of inhibiting the growth of cancer cells expressing a NY-ESO-1 or LAGE-1a protein. The method comprises exposing the cancer cells to a genetically engineered cell described herein that is capable of inhibiting the growth of the cancer cells.

In another aspect, the invention provides a method of treating or preventing cancer in a subject. The method comprises administering to the subject autologous genetically modified T-cells (i) expressing an α-chain of a T-cell receptor described herein, (ii) expressing a β-chain of a T-cell receptor described herein, or (iii) expressing a T-cell receptor described herein, and/or (iv) transduced with one or more of the nucleic acids or the recombinant expression vectors described herein, in an amount effective to treat or prevent cancer in the subject.

In another aspect, the invention provides a method for treating or preventing cancer in a subject. The method comprises the steps of (i) extracting T-cells from the subject; (ii) introducing into the T-cells one or more nucleic acids or one or more of the recombinant vectors described herein; and (iii) administering the T-cells produced by step (ii) to the subject.

### BRIEF DESCRIPTION OF THE DRAWINGS

The foregoing and other objects, features and advantages of the invention will become apparent from the following description of preferred embodiments, as illustrated in the accompanying drawings, in which:

FIG. 1 shows the recognition of the HLA-A2 restricted NY-ESO-1:157-165 epitope (“ESOp157-165”) of SEQ ID NO: 1 by CD8⁺ T-cell lines using IFN-γ and GM-CSF release as an indicator of target recognition. FIG. 1A depicts the identification of a T cell clone (806) recognizing HLA-A2/NY-ESO-1+ tumor cells. The 806 T-cells were shown to recognize the NY-ESO-1:157-165 peptide presented by HLA-A2+ 1088-B cells (FIG. 1A, left). The 806 T-cells were shown to recognize the NY-ESO-1 protein, but not GFP, when introduced into cosA2 presenting cells (FIG. 1A, left). The 806 T-cells were able to recognize the 624.38 melanoma line (HLA-A2+/NY-ESO-1+), but not the variant 624.28 melanoma cell line (HLA-A2−/NY-ESO-1+) (FIG. 1A, left). Titration studies measuring GM-CSF release at the indicated concentrations displayed a high avidity of the TCR for NY-ESO-1:157-165 (FIG. 1A, right). FIG. 1B depicts the discovery of a single TCR up pair recognizing NY-ESO-1:157-165. Jurkat76 (J76) cells were transduced with various a and β chain combinations obtained from the TCR sequencing results of the 806 T cell clone. Only the α1β1 combination demonstrated a specific response against NY-ESO-1:157-165-pulsed T2 cells. FIG. 1C depicts recognition of HLA-A2/NY-ESO-1:157-165 pentamers by 806α1β1. J76 cells transduced with either a control TCR or the 806α1β1 TCR construct were analyzed by flow cytometry for binding to a NY-ESO-1:157-165-pentamer. Only the 806α1β1 TCR demonstrated binding to the NY-ESO-1 pentamer. Antibodies against the mouse C terminal domain were used to assess the cell-surface expression of the TCR heterodimers. FIG. 1D depicts tumor cell recognition by the 806TCR. Transduced T cells (black), as opposed to untransduced cells (dark grey) or target cells alone (light grey), showed specific activation when exposed to a panel of A2+/NYESO1+ cells (Colo-205-NYESO1, FM-6, FM-82, HEPG2-NYESO, SK-MEL-37, UACC-257, and MEL-624.38); while they showed no specific activity when exposed to A2−/NYESO1− cells (HpAF-II, LS174T, LS714T, and SK-LU-1) or A2+/NYESO1− cells (SK-LU-1-NYESO, MEL-624.28, A549, Colo-205, Cos-7-A2, HepG-2, Kato-III, and SK-MEL-23). FIG. 1E depicts cell killing by the 806TCR. 806TCR transduced T cells showed the ability kill A2+/NY-ESO-1+ cells (MEL-624.38, FM-6, UACC-257) but not A2−/NY-ESO-1+ target cells (MEL-624.28) after culturing for 24 hours. Untransduced T cells demonstrated no killing activity.

FIG. 2 depicts the α-chain amino acid sequence (SEQ ID NO: 2; FIG. 2A), α-chain codon-optimized nucleotide sequence (SEQ ID NO: 100; FIG. 2B), β1-chain amino acid sequence (SEQ ID NO: 95; FIG. 2C), and β1-chain codon-optimized nucleotide sequence (SEQ ID NO: 102; FIG. 2D) of the 806 TCR including human α- and β-chain constant regions. The CDR regions are in red and are underlined.

FIGS. 3A and 3B depict the amino acid sequence of (SEQ ID NO: 14), and the polynucleotide sequence encoding (SEQ ID NO: 27), the 806 TCR α-P2A-β1 fusion protein with the P2A sequence shown in bold. FIGS. 3C and 3D depict the amino acid sequence of (SEQ ID NO: 29), and the polynucleotide sequence encoding (SEQ ID NO: 30), the 806 TCR α-P2A-β1-T2A-CD34t fusion protein with the P2A sequence bolded, the T2A sequence bolded and underlined, and the CD34t sequence in lower case.

FIG. 4 shows sequence alignments of amino acid sequences for the 806 T-cell receptor against sequences for other T-cell receptors that are able to recognize the NY-ESO-1:157-165 peptide. The 806 T-cell receptor α-chain amino acid sequence (FIG. 4A) and the 806 T-cell receptor β1-chain amino acid sequence (FIG. 4B) were aligned with sequences from other TCR chains identified as 1G4 (as described in U.S. Patent Application Publication No. US2009/053184), BC1 (as described in International Patent Application Publication No. WO2020/188348). 1G4C113 (as described in International Patent Application Publication No. WO2005/113595), UC-1E4 (as described in International Patent Application Publication No. WO2020/(086158). V17 and V12-4 (both as described in International Patent Application Publication No. WO2017/076308). For the T-cell receptor sequences, the CDR regions are bolded and in red and constant regions are underlined. FIG. 4A discloses SEQ ID Nos. 108-112 and SEQ ID NO: 37, respectively, in order of appearance. FIG. 4B discloses SEQ ID Nos. 113, 39, and 114-117, respectively, in order of appearance.

FIG. 5 depicts sequences of the 806 T-cell receptor (with human constant regions) with exemplary glycosylation sites mutated. The amino acid and polynucleotide sequences of the 806 T-cell receptor α-chain with exemplary mutations (SEQ ID NOS: 75 and 76, respectively; FIGS. 5A and 5B), and the 806 T-cell receptor β1-chain with exemplary mutations (SEQ ID NOS: 97 and 98, respectively; FIGS. 5C and 5D) are depicted. The amino acid mutations and corresponding nucleotide sequence changes are shown as underlined. The constant regions are shown in bold.

FIG. 6 depicts the amino acid and polynucleotide sequences of 806 T-cell receptor α-chain (SEQ ID NOS: 37 and 38, respectively; FIGS. 6A-6B) and the β1-chain (SEQ ID NOS: 39 and 40, respectively; FIGS. 6C-6D) with the constant regions replaced by those from a known murine TCR. The constant regions are shown in bold.

FIG. 7 depicts sequences of the 806 T-cell receptor (with murine constant regions) with exemplary glycosylation sites mutated. The amino acid and polynucleotide sequences of the 806 T-cell receptor α-chain with exemplary mutations (SEQ ID NOS: 79 and 80, respectively; FIGS. 7A and 7B), and the 806 T-cell receptor β1-chain with exemplary mutations (SEQ ID NOS: 81 and 82, respectively; FIGS. 7C and 7D) are depicted. The amino acid mutations and corresponding nucleotide sequence changes are shown as underlined. The constant regions are shown in bold.

FIG. 8 is a line graph showing 806TCR-T cell activity (as measured by interferon-γ release) as a function of NY-ESO-1157-165 peptide concentration. HLA-A*02:01+ antigen presenting cells were pulsed with NY-ESO-1:157-165 peptide at the indicated concentration, co-cultured with 806TCR-T cells, and interferon-γ release was measured by ELISA.

FIG. 9 depicts real-time fluorescent images of target MEL-624.38 cells incubated with the indicated 806TCR-T cells (or controls) at the indicated time points. Columns represent co-culture conditions: no T cells, donor 1 transduced 806TCR-T cells, donor 2 transduced 806TCR-T cells, and 10% DMSO (v/v) (dead cell control). Rows represent timepoints when images were taken: day 0 (0 hrs:12 min), day 2 (48 hrs:12 mins), and day 3 (72 hours:12 mins).

FIG. 10 depicts line graphs showing cell nuclei count over time (as measured by fluorescent microscopy) following incubation of non-target MEL-624.28 cells (FIG. 10A) or target MEL-624.38 cells (FIG. 10B) with 806TCR-T cells. Green lines represent target cells only (without T cells). Red lines depict dead cell control of 10% DMSO (v/v). Blue lines depict co-culture with 806TCR-T cells. Results are normalized to time 0 (0 hrs:12 mins).

FIG. 11 is a bar graphic depicting 806TCR-T cell activity (as measured by interferon-γ release) following co-culture with a panel of normal (non-cancerous) cells and one target cancerous cell line. UT indicates untransduced. D1 and D2 refer to different donors for pulmonary fibroblasts. D882, D081, and D200 refer to different donors for T cells.

FIG. 12 depicts binding of the 1G4LY TCR (FIG. 12A) and 806 TCR (FIG. 12B) to peptide/pMHC1 complex (biotin-HLA-A*02:01-SLLMWITQC (SEQ ID NO: 1)) as observed by surface plasmon resonance. Each trace corresponds to the indicated concentration of TCR. Results were used for the calculation of binding kinetics, as described in Example 7.

### DETAILED DESCRIPTION OF THE INVENTION

The present invention provides T-cell receptors, as well as fragments and variants thereof, that bind to the NY-ESO-1 and/or the LAGE-1a antigens expressed on the surface of cancer cells in an HLA-restricted manner. An exemplary T-cell receptor of the invention is immunoreactive with the NY-ESO-1 and/or LAGE-1a epitope SLLMWITQC (SEQ ID NO: 1) and/or SLLMWITQCFL (SEQ ID NO: 28). The binding can be in association with recognition of HLA-A2 restricted antigens. For example, the binding can be restricted to HLA-A*0201, HLA-A*0202 or potentially other HLA-A2 subtype-expressing cells. The expression of NY-ESO-1 and/or LAGE-1a on the surface of cancer cells provides a target for specific binding of the T-cell receptor, as well as fragments and variants thereof, to the surface of cancer cells. As used herein, the term “immunoreactive” is understood to mean that a T-cell receptor, for example, a T-cell receptor of the invention, as well as fragments and variants thereof, can bind specifically to an epitope present in an antigen, for example, an NY-ESO-1 antigen or a LAGE-1a antigen, optionally in a MHC-restrictive manner.

### I. T-Cell Receptors

The present invention provides an isolated recombinant T-cell receptor (for example, a non-naturally occurring T-cell receptor). In certain embodiments, the T-cell receptor comprises an α-chain and β-chain wherein the α-chain and β-chain variable regions define a binding site for binding to the NY-ESO-1 and/or LAGE-1a epitope SLLMWITQC (SEQ ID NO: 1) and/or SLLMWITQCFL (SEQ ID NO: 28) in an HLA-A2 restricted manner. In certain embodiments, the T-cell receptor comprises an α-chain and β-chain wherein the α-chain and β-chain variable regions define a binding site for binding to the NY-ESO-1 and/or LAGE-1a epitope SLLMWITQC (SEQ ID NO: 1) in an HLA-A2 restricted manner.

An exemplary TCR is characterized as follows. The full length α-chain and β-chain of the T-cell receptor comprise SEQ ID NOs: 2 and 95, respectively. The α-chain variable region comprises the amino acid of SEQ ID NO: 3, while the β-chain variable region comprises the amino acid sequence of SEQ ID NO: 9. The α-chain constant region comprises the amino acid sequence of SEQ ID NO: 4 and the β-chain constant region comprises the amino acid sequence of SEQ ID NO: 10. Each of the α-chain and β-chain variable regions comprises three CDR regions wherein the α-chain CDRs comprise the amino acid sequences of SEQ ID NO: 5 (CDR₁), SEQ ID NO: 6 (CDR₂) and SEQ ID NO: 7 (CDR₃) and the β-chain CDRs comprise the amino acid sequences of SEQ ID NO: 11 (CDR₁), SEQ ID NO: 12 (CDR₂) and SEQ ID NO: 13 (CDR₃).

An additional exemplary TCR is characterized as follows. The full length α-chain and β-chain of the T-cell receptor comprise SEQ ID NO: 87 and 99, respectively. The α-chain variable region comprises the amino acid of SEQ ID NO: 83, while the β-chain variable region comprises the amino acid sequence of SEQ ID NO: 85. The α-chain constant region comprises the amino acid sequence of SEQ ID NO: 4 and the β-chain constant region comprises the amino acid sequence of SEQ ID NO: 10. Each of the α-chain and β-chain variable regions comprises three CDR regions wherein the α-chain CDRs comprise the amino acid sequences of SEQ ID NO: 5 (CDR₁), SEQ ID NO: 6 (CDR₂) and SEQ ID NO: 7 (CDR₃) and the β-chain CDRs comprise the amino acid sequences of SEQ ID NO: 11 (CDR₁), SEQ ID NO: 12 (CDR₂) and SEQ ID NO: 13 (CDR₃).

For clarity, certain sequences are set forth in TABLE 1.

TABLE 1

| | | |
|---|---|---|
| SEQ ID | Type | Description |

TCR Sequences

| | | |
|---|---|---|
| 2 | Protein | α chain full length |
| | | (human constant region) |
| 87 | Protein | α chain full length |
| | | (human constant region) |
| 3 | Protein | α chain variable region |
| 83 | Protein | α chain variable region |
| 4 | Protein | α chain constant region |
| | | (human) |
| 5 | Protein | α chain CDR₁ |
| 6 | Protein | α chain CDR₂ |
| 7 | Protein | α chain CDR₃ |
| 95 | Protein | β chain full length |
| | | (human constant region) |
| 99 | Protein | β chain full length |
| | | (human constant region) |
| 9 | Protein | β chain variable region |
| 85 | Protein | β chain variable region |
| 10 | Protein | β chain constant region |
| | | (human) |
| 11 | Protein | β chain CDR₁ |
| 12 | Protein | β chain CDR₂ |
| 13 | Protein | β chain CDR₃ |
| 100 | Nucleic Acid | α chain full length |
| | | (human constant region) |
| 15 | Nucleic Acid | α chain full length |
| | | (human constant region) |
| 101 | Nucleic Acid | α chain variable region |
| 16 | Nucleic Acid | α chain variable region |
| 17 | Nucleic Acid | α chain constant region |
| | | (human) |
| 18 | Nucleic Acid | α chain CDR₁ |
| 19 | Nucleic Acid | α chain CDR₂ |
| 20 | Nucleic Acid | α chain CDR₃ |
| 102 | Nucleic Acid | β chain full length |
| | | (human constant region) |
| 96 | Nucleic Acid | β chain full length |
| | | (human constant region) |
| 103 | Nucleic Acid | β chain variable region |
| 22 | Nucleic Acid | β chain variable region |
| 23 | Nucleic Acid | β chain constant region |
| | | (human) |
| 24 | Nucleic Acid | β chain CDR₁ |
| 25 | Nucleic Acid | β chain CDR₂ |
| 26 | Nucleic Acid | β chain CDR₃ |

Binding Epitopes

| | | |
|---|---|---|
| 1 | Protein | NY-ESO-1: 157-165 |
| 28 | Protein | NY-ESO-1: 157-167 |

Single chain TCRs

| | | |
|---|---|---|
| 14 | Protein | 806 αP2Aβ1 bi-cistron |
| 27 | Nucleic Acid | 806 αP2Aβ1 bi-cistron |
| 29 | Protein | 806 αP2AβT2ACD34t |
| | | tri-cistron |
| 30 | Nucleic Acid | 806 αP2AβT2ACD34t |
| | | tri-cistron |
| 93 | Protein | 806 αP2AβT2ACD34t |
| | | tri-cistron |
| 94 | Nucleic Acid | 806 αP2AβT2ACD34t |
| | | tri-cistron |

TCR Variants/Engineered TCRs

| | | |
|---|---|---|
| 75 | Protein | α chain full length |
| | | (human constant region) N→Q |
| 76 | Nucleic Acid | α chain full length |
| | | (human constant region) N→Q |
| 97 | Protein | β chain full length |
| | | (human constant region) N→Q |
| 98 | Nucleic Acid | β chain full length |
| | | (human constant region) N→Q |
| 37 | Protein | α chain full length |
| | | (murine constant region) |
| 84 | Protein | α chain full length |
| | | (murine constant region) |
| 38 | Nucleic Acid | α chain full length |
| | | (murine constant region) |
| 49 | Protein | α chain constant |
| | | region (murine) |
| 50 | Nucleic Acid | α chain constant |
| | | region (murine) |
| 39 | Protein | β chain full length |
| | | (murine constant region) |
| 86 | Protein | β chain full length |
| | | (murine constant region) |
| 40 | Nucleic Acid | β chain full length |
| | | (murine constant region) |
| 51 | Protein | β chain constant |
| | | region (murine) |
| 52 | Nucleic Acid | β chain constant |
| | | region (murine) |
| 79 | Protein | α chain full length |
| | | (murine constant region) N→Q |
| 80 | Nucleic Acid | α chain full length |
| | | (murine constant region) N→Q |
| 81 | Protein | β chain full length |
| | | (murine constant region) N→Q |
| 82 | Nucleic Acid | β chain full length |
| | | (murine constant region) N→Q |

An additional exemplary TCR is characterized as follows. The full length α-chain and β-chain of the T-cell receptor comprise SEQ ID NO: 87 and 99, respectively. The α-chain variable region comprises the amino acid of SEQ ID NO: 83, while the β-chain variable region comprises the amino acid sequence of SEQ ID NO: 85. The α-chain constant region comprises the amino acid sequence of SEQ ID NO: 4 and the β-chain constant region comprises the amino acid sequence of SEQ ID NO: 10. Each of the α-chain and β-chain variable regions comprises three CDR regions wherein the α-chain CDRs comprise the amino acid sequences of SEQ ID NO: 5 (CDR₁), SEQ ID NO: 6 (CDR₂) and SEQ ID NO: 7 (CDR₃) and the β-chain CDRs comprise the amino acid sequences of SEQ ID NO: 11 (CDR₁), SEQ ID NO: 12 (CDR₂) and SEQ ID NO: 13 (CDR₃).

An additional exemplary TCR is characterized as follows. The full length α-chain and β-chain of the T-cell receptor comprise SEQ ID NO: 53 and 56, respectively. The α-chain variable region comprises the amino acid of SEQ ID NO: 54, while the β-chain variable region comprises the amino acid sequence of SEQ ID NO: 57. The α-chain constant region comprises the amino acid sequence of SEQ ID NO: 55 and the β-chain constant region comprises the amino acid sequence of SEQ ID NO: 58. Each of the α-chain and β-chain variable regions comprises three CDR regions wherein the α-chain CDRs comprise the amino acid sequences of SEQ ID NO: 43 (CDR₁), SEQ ID NO: 44 (CDR₂) and SEQ ID NO: 45 (CDR₃) and the β-chain CDRs comprise the amino acid sequences of SEQ ID NO: 46 (CDR₁), SEQ ID NO: 47 (CDR₂) and SEQ ID NO: 48 (CDR₃).

In one aspect, the invention provides an isolated, recombinant α-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). In another aspect, the invention provides an isolated, recombinant α-chain of a T-cell receptor immunoreactive with an epitope of a NY-ESO-1 and/or LAGE-1a protein comprising the amino acid sequence SLLMWITQC (SEQ ID NO: 1). The α-chain comprises one or more of the following amino acid sequences: (i) an amino acid sequence of SEQ ID NO: 2 or SEQ ID NO: 87, or an amino acid sequence having greater than 97%, 98% or 99% identity to the amino acid sequence of SEQ ID NO: 2 or SEQ ID NO: 87; (ii) an α-chain variable region amino acid sequence of SEQ ID NO: 3 or SEQ ID NO: 83, or an amino acid sequence having greater than 96%, 97%, 98% or 99% identity to the amino acid sequence of SEQ ID NO: 3 or SEQ ID NO: 83; (iii) an α-chain CDR₃ amino acid sequence of SEQ ID NO: 7; (iv) an α-chain CDR₁ amino acid sequence of SEQ ID NO: 5; and (v) an α-chain CDR₂ amino acid sequence of SEQ ID NO: 6.

In another aspect, the invention provides an isolated recombinant β-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). In another aspect, the invention provides an isolated, recombinant β-chain of a T-cell receptor immunoreactive with an epitope of a NY-ESO-1 and/or LAGE-1a protein comprising the amino acid sequence SLLMWITQC (SEQ ID NO: 1). The β-chain comprises one or more of the following amino acid sequences: (i) an amino acid sequence of SEQ ID NO: 8, SEQ ID NO: 88, SEQ ID NO: 95, or SEQ ID NO: 99, or an amino acid sequence having greater than 97%, 98% or 99% identity to the amino acid sequence of SEQ ID NO: 8, SEQ ID NO: 88, SEQ ID NO: 95, or SEQ ID NO: 99; (ii) a β-chain variable region amino acid sequence of SEQ ID NO: 9 or SEQ ID NO: 85, or an amino acid sequence having greater than 97%, 98%, or 99% identity to the amino acid sequence of SEQ ID NO: 9 or SEQ ID NO: 85; (iii) a β-chain CDR₃ amino acid sequence of SEQ ID NO: 13; (iv) a β-chain CDR₁ amino acid sequence of SEQ ID NO: 11; and (v) a β-chain CDR₂ amino acid sequence of SEQ ID NO: 12.

In another aspect, the invention provides a recombinant T-cell receptor immunoreactive with a SLLMWITQC (SEQ ID NO: 1) and/or SLLMWITQCFL (SEQ ID NO: 28) epitope comprising at least one of the foregoing α-chains and at least one of the foregoing β-chains.

In another aspect, the invention provides an isolated, recombinant T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). In another aspect, the invention provides an isolated, recombinant T-cell receptor immunoreactive with an epitope of a NY-ESO-1 and/or LAGE-1a protein comprising the amino acid sequence SLLMWITQC (SEQ ID NO: 1).

In certain embodiments, the T-cell receptor comprises an α-chain and a β-chain each comprising a CDR₁, CDR₂, and a CDR₃, wherein the α-chain CDR₃ comprises the amino acid sequence of SEQ ID NO: 7, and the β-chain CDR₃ comprises the amino acid sequence of SEQ ID NO: 13. Optionally, or in addition, the T-cell receptor α-chain CDR₁ comprises the amino acid sequence of SEQ ID NO: 5, and the β-chain CDR₁ comprises the amino acid sequence of SEQ ID NO: 11. Optionally, or in addition, the T-cell receptor α-chain CDR₂ comprises the amino acid sequence of SEQ ID NO: 6, and the β-chain CDR₂ comprises the amino acid sequence of SEQ ID NO: 12.

In certain embodiments, the T-cell receptor comprises an α-chain and a β-chain each comprising a CDR₁, CDR₂, and a CDR₃, wherein the α-chain CDR₃ comprises the amino acid sequence of SEQ ID NO: 45, and the β-chain CDR₃ comprises the amino acid sequence of SEQ ID NO: 48. Optionally, or in addition, the T-cell receptor α-chain CDR₁ comprises the amino acid sequence of SEQ ID NO: 43, and the β-chain CDR₁ comprises the amino acid sequence of SEQ ID NO: 46. Optionally, or in addition, the T-cell receptor α-chain CDR₂ comprises the amino acid sequence of SEQ ID NO: 44, and the β-chain CDR₂ comprises the amino acid sequence of SEQ ID NO: 47.

In certain embodiments, the T-cell receptor comprises an α-chain variable region comprising an amino acid sequence of SEQ ID NO: 3 or SEQ ID NO: 83, or an amino acid sequence having greater than 96%, 97%, 98%, or 99% identity to the amino acid sequence of SEQ ID NO: 3 or SEQ ID NO: 83; and a β-chain variable region comprising an amino acid sequence of SEQ ID NO: 9 or SEQ ID NO: 85, or an amino acid an amino acid sequence having greater than 97%, 98%, or 99% identity to the amino acid sequence of SEQ ID NO: 9 or SEQ ID NO: 85. Optionally, or in addition, the T-cell receptor α-chain comprises the amino acid sequence of SEQ ID NO: 2 or SEQ ID NO: 87, or an amino acid sequence having greater than 97%, 98%, or 99% identity to the amino acid sequence of SEQ ID NO: 2 or SEQ ID NO: 87, and/or the β-chain comprises the amino acid sequence of SEQ ID NO: 8, SEQ ID NO: 88, SEQ ID NO: 95, or SEQ ID NO: 99, or an amino acid sequence having greater than 97%, 98% or 99% identity to the amino acid sequence of SEQ ID NO: 8, SEQ ID NO: 88, SEQ ID NO: 95, or SEQ ID NO: 99.

In certain embodiments, the T-cell receptor comprises an α-chain variable region comprising an amino acid sequence of SEQ ID NO: 54 or an amino acid sequence having greater than 96%, 97%, 98%, or 99% identity to the amino acid sequence of SEQ ID NO: 54; and/or a β-chain variable region comprising an amino acid sequence of SEQ ID NO: 57 or an amino acid an amino acid sequence having greater than 97%, 98%, or 99% identity to the amino acid sequence of SEQ ID NO: 57. Optionally, or in addition, the T-cell receptor α-chain comprises the amino acid sequence of SEQ ID NO: 53 or an amino acid sequence having greater than 97%, 98%, or 99% identity to the amino acid sequence of SEQ ID NO: 53, and/or the β-chain comprises the amino acid sequence of SEQ ID NO: 56 or an amino acid sequence having greater than 97%, 98% or 99% identity to the amino acid sequence of SEQ ID NO: 56.

In each of the foregoing aspects, the T-cell receptor is optionally a single chain T-cell receptor, optionally where the α-chain is linked to the β-chain via an amino acid linker. For example, in one embodiment, the isolated T-cell receptor can comprise the amino acid sequence of SEQ ID NO: 14 or SEQ ID NO: 29, which can be encoded by the polynucleotide sequence of SEQ ID NO: 27 or SEQ ID NO: 30, respectively.

Furthermore, in certain embodiments of the foregoing aspects, the T-cell receptor is immunoreactive with the epitope SLLMWITQC (SEQ ID NO: 1) and/or SLLMWITQCFL (SEQ ID NO: 28) in an HLA-A2 restricted manner. For example, the immunoreactivity to the epitope SLLMWITQC (SEQ ID NO: 1) and/or SLLMWITQCFL (SEQ ID NO: 28) can be (i) HLA-A*0201 or (ii) HLA-A*0202 restricted. The T-cell receptor may potentially be restricted by other HLA-A2 subtypes, and other HLA class I molecules.

It is contemplated that, for each of the amino acid sequences provided herein, the sequences optionally include at least 1, 2, 3, 4, 5, 6, 7, 8, 9, 10, 15, or 20 amino acid substitutions not present at a given position. For example, contemplated herein are amino acid sequences having at least 1, 2, 3, 4, 5, 6, 7, 8, 9, 10, 15, or 20 amino acid substitutions not present in SEQ ID NOs: 2, 3, 4, 8, 9, 10, 83, 85, 87, 88, 95, or 99.

It is contemplated that included within the scope of the invention, are functional variants of a disclosed T-cell receptor. As used herein, the term “functional variant” is understood to mean a T-cell receptor α- and/or β-chain having substantial or significant sequence identity or similarity to the T-cell receptor α- and/or β-chain of the invention as described above, wherein said functional variants retain the ability to specifically bind (for example, avidity, affinity, association constant and/or dissociation constant) to an epitope (for example, an epitope of a NY-ESO-1 and/or LAGE-1a protein comprising the amino acid sequence SLLMWITQC (SEQ ID NO: 1) and/or SLLMWITQCFL (SEQ ID NO: 28)), to a similar extent (for example, greater than 50%, 60%, 70%, 80%, 90% or 95%) of the T-cell receptor described herein. Such functional variants include polypeptides with partial sequence identity, peptides having one or more specific conservative and/or non-conservative amino acid substitutions.

Functional variants of the invention can, for example, comprise the amino acid sequence of the T-cell receptor as described above, as well as fragments thereof, but which have at least one conservative amino acid substitution. Conservative amino acid substitutions are known in the art, and include amino acid substitutions in which one amino acid having certain physical and/or chemical properties is exchanged for another amino acid that has the same chemical or physical properties. Alternatively or additionally, the functional variants can comprise the amino acid sequence of the T-cell receptor of the invention with at least one non-conservative amino acid substitution wherein said non-conservative amino acid substitution does not interfere with or inhibit the biological activity of the functional variant.

The functional variants can be assayed in a number of different ways including the approaches set forth in the Examples. For example, target cells expressing NY-ESO-1 and/or LAGE-1a can be contacted with genetically engineered T-cells expressing the variant T-cell receptor. Release assays may then be conducted to detect the presence of IFN-γ or GM-CSF in culture media indicating recognition of NY-ESO-1 and/or LAGE-1a by the engineered T-cells and their subsequent activation.

In certain embodiments, a T-cell receptor that binds to NY-ESO-1 and/or LAGE-1a comprises an α-chain variable region that has at least 75%, 80%, 85%, 90%, 95%, 96%, 97%, 98%, 99% identity to the α-chain variable region of SEQ ID NO: 3 or SEQ ID NO: 83. Optionally, or in addition, the T-cell receptor that binds to NY-ESO-1 and/or LAGE-1a comprises a β-chain variable region that has at least 75%, 80%, 85% 90%, 95%, 96%, 97%, 98%, 99% identity to the β-chain variable region of SEQ ID NO: 9 or SEQ ID NO: 85.

In certain embodiments, a T-cell receptor that binds to NY-ESO-1 and/or LAGE-1a comprises an α-chain variable region that has at least 75%, 80%, 85%, 90%, 95%, 96%, 97%, 98%, 99% identity to the α-chain variable region of SEQ ID NO: 2 or SEQ ID NO: 87. Optionally, or in addition, the T-cell receptor that binds to NY-ESO-1 and/or LAGE-1a comprises a β-chain variable region that has at least 75%, 80%, 85% 90%, 95%, 96%, 97%, 98%, 99% identity to the β-chain variable region of SEQ ID NO: 8, SEQ ID NO: 88, SEQ ID NO: 95, or SEQ ID NO: 99.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SC. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

The invention further comprises proteins or polypeptides comprising one or more functional fragments of a T-cell receptor of the invention. With respect to such proteins or polypeptides, the functional fragment can be any fragment comprising amino acids of a T-cell receptor, including α- and β-chains, or functional variants thereof, of which it is a part, provided that the functional fragment specifically binds to NY-ESO-1 and/or LAGE-1a. The term “functional fragment” when used in reference to a T-cell receptor, or functional variants thereof, refers to any part or fragment of the T-cell receptor, or functional variant thereof, which retains at least 50%, 60%, 70%, 80%, 85%, 90%, 95%, 96%, 97%, 98% or 99% of one or more biological activities of the T-cell receptor of the invention. Functional fragments encompass, for example, those parts of a T-cell receptor, or functional variants thereof, that retain the ability to specifically bind to NY-ESO-1 and/or LAGE-1, or detect, treat, or prevent cancer, to a similar extent, the same extent, or to a higher extent, as a T-cell receptor of the invention, or functional variants thereof.

The functional fragment may comprise additional amino acids at the amino and/or carboxy termini of the fragment wherein the additional amino acids do not interfere with the biological function of the functional fragment, e.g., binding to the NY-ESO-1 and/or LAGE-1a antigen. In a preferred embodiment, the additional amino acids can be added to the functional fragment to provide a peptide tag to aid in the purification of a T-cell receptor or to enhance the biological activity of the T-cell receptor.

### II. Engineered T-cell Receptors

The invention also provides engineered or modified T-cell receptors that retain the ability to bind to the NY-ESO-1 and/or LAGE-1a antigen. Such T-cell receptors include, for example, one or more point mutations, insertions, and/or deletions. Other engineered T-cell receptors include, for example, single chain fusions proteins, bispecific proteins, chimeric T-cell receptors and T-cell receptors associated with a detectable label or an effector therapeutic agent.

In one embodiment of the invention, the invention provides a modified T-cell receptor of the invention in the form of a single chain fusion protein comprising a linker peptide linking the α-chain of SEQ ID NO: 2 or SEQ ID NO: 87, and the β-chain of SEQ ID NO: 8, SEQ ID NO: 88, SEQ ID NO: 95, or SEQ ID NO: 99, as well as fragments and functional variants of said α- and β-chains.

The T-cell receptor α-chain may comprise one or more of the following amino acid sequences: (i) an amino acid sequence of SEQ ID NO: 2 or SEQ ID NO: 87, or an amino acid sequence having greater than 97%, 98%, or 99% identity to the amino acid sequence of SEQ ID NO: 2 or SEQ ID NO: 87; (ii) an α-chain variable region amino acid sequence of SEQ ID NO: 3 or SEQ ID NO: 83, or an amino acid sequence having greater than 96%, 97%, 98%, or 99% identity to the amino acid sequence of SEQ ID NO: 3 or SEQ ID NO: 83; (iii) an α-chain CDR3 amino acid sequence of SEQ ID NO: 7; (iv) an α-chain CDR₁ amino acid sequence of SEQ ID NO: 5; and (v) an α-chain CDR2 amino acid sequence of SEQ ID NO: 6. The T-cell receptor β-chain may comprise one or more of the following amino acid sequences: (i) an amino acid sequence of SEQ ID NO: 8, SEQ ID NO: 88, SEQ ID NO: 95, or SEQ ID NO: 99, or an amino acid sequence having greater than 97% identity to the amino acid sequence of SEQ ID NO: 8, SEQ ID NO: 88, SEQ ID NO: 95, or SEQ ID NO: 99; (ii) a β-chain variable region amino acid sequence of SEQ ID NO: 9 or SEQ ID NO: 85, or an amino acid sequence having greater than 97% identity to the amino acid sequence of SEQ ID NO: 9 or SEQ ID NO: 85; (iii) a β-chain CDR₃ amino acid sequence of SEQ ID NO: 13; (iv) a β-chain CDR₁ amino acid sequence of SEQ ID NO: 11; and (v) β-chain CDR₂ amino acid sequence of SEQ ID NO: 12.

Furthermore, the single chain T-cell receptor can comprise α- and β-chains each comprising a CDR₁, CDR₂, and a CDR₃, wherein the α-chain CDR₃ comprises the amino acid sequence of SEQ ID NO: 7, and the β-chain CDR₃ comprises the amino acid sequence of SEQ ID NO: 13. The T-cell receptor may further comprise the α-chain CDR₁ of SEQ ID NO: 5 and the β-chain CDR₁ of SEQ ID NO: 11 and/or the α-chain CDR₂ of SEQ ID NO: 6 and the β-chain CDR₂ of SEQ ID NO: 12.

In another embodiment, the single chain T-cell receptor comprises an α-chain variable region comprising an amino acid sequence of SEQ ID NO: 3 or SEQ ID NO: 83, or an amino acid sequence having greater than 96%, 97%, 98%, or 99% identity to the amino acid sequence of SEQ ID NO: 3 or SEQ ID NO: 83; and a β-chain variable region comprising an amino acid sequence of SEQ ID NO: 9 or SEQ ID NO: 85, or an amino acid an amino acid sequence having greater than 97%, 98%, or 99% identity to the amino acid sequence of SEQ ID NO: 9 or SEQ ID NO: 85.

A single chain T-cell receptor is also provided, wherein the α-chain comprises the amino acid sequence of SEQ ID NO: 2 or SEQ ID NO: 87, or an amino acid sequence having greater than 97%, 98%, or 99% identity to the amino acid sequence of SEQ ID NO: 2 or SEQ ID NO: 87, and the β-chain comprises the amino acid sequence of SEQ ID NO: 8, SEQ ID NO: 88, SEQ ID NO: 95, or SEQ ID NO: 99, or an amino acid sequence having greater than 97%, 98%, 99% identity to the amino acid sequence of SEQ ID NO: 8, SEQ ID NO: 88, SEQ ID NO: 95, or SEQ ID NO: 99. In a specific embodiment, the invention provides a single chain T-cell receptor having the amino acid sequence of SEQ ID NO: 14. In a specific embodiment, the invention provides a single chain T-cell receptor having the amino acid sequence of SEQ ID NO: 29. In a specific embodiment, the invention provides a single chain T-cell receptor having the amino acid sequence of SEQ ID NO: 89. In a specific embodiment, the invention provides a single chain T-cell receptor having the amino acid sequence of SEQ ID NO: 91. In a specific embodiment, the invention provides a single chain T-cell receptor having the amino acid sequence of SEQ ID NO: 93.

It is contemplated that, for each of the amino acid sequences provided herein, the sequences optionally include at least 1, 2, 3, 4, 5, 6, 7, 8, 9, 10, 15, or 20 amino acid substitutions (mutations) not present at a given position. For example, contemplated herein are amino acid sequences having at least 1, 2, 3, 4, 5, 6, 7, 8, 9, 10, 15, or 20 amino acid substitutions not present in SEQ ID NOs: 2, 3, 4, 8, 9, 10, 83, 85, 87, 88, 95, or 99. Mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see Sambrook & Russell, (2001) Molecular Cloning-A Laboratory Manual (3rd Ed.) CSHL Press. For further information on LIC procedures see Rashtchian (1995) CURR. OPIN. BIOTECHNOL. 6 (1): 30-6.

Mutagenesis of the T-cell receptors described herein may be performed to increase, for example, the affinity, specificity, membrane targeting, half-life and expression levels of a T-cell receptor, which can be beneficial in clinical applications (Abate-Daga et al. (2014) PLoS ONE 9:e93321; Udyavar et al. (2009) J. IMMUNOL. 182:4439-4447; Kuball et al. (2009) J. EXP. MED. 206:463-475). Mutants of a T-cell receptor may comprise amino acid additions, deletions and/or insertions. The mutations may be concentrated in one or more regions such as constant regions, framework regions or variable regions, including the CDR variable regions of the α- and/or β-chains, or they may be spread throughout the molecule. The variants may be recombinantly or synthetically produced.

The present invention provides T-cell chimeric proteins where one or more regions of a human T-cell receptor of the invention are replaced with corresponding T-cell receptor regions derived from species other than human, such as a pig or rodent (for example, a rat or mouse). In one embodiment, the human constant regions of the α- and/or β-chains are replaced with known murine T-cell receptor constant regions. The pairing between murine TCR constant regions may reduce the mispairing of transduced T-cell receptors with endogenous T-cell receptors in a human subject.

Exemplary human α-chain constant regions are depicted in SEQ ID NOs: 4 and 55. Accordingly, in certain embodiments, a T-cell receptor that binds to NY-ESO-1 and/or LAGE-1a comprises SEQ ID NO: 4 or SEQ ID NO: 55, or an amino acid sequence that has at least 75%, 80%, 85%, 90%, 95%, 96%, 97%, 98%, or 99% identity to SEQ ID NO: 4 or SEQ ID NO: 55. An exemplary murine α-chain constant region is depicted in SEQ ID NO: 49. Accordingly, in certain embodiments, a T-cell receptor that binds to NY-ESO-1 and/or LAGE-1a comprises SEQ ID NO: 49, or an amino acid sequence that has at least 75%, 80%, 85%, 90%, 95%, 96%, 97%, 98%, or 99% identity to SEQ ID NO: 49. Exemplary human β-chain constant regions are depicted in SEQ ID NOs: 10 and 58. Accordingly, in certain embodiments, a T-cell receptor that binds to NY-ESO-1 and/or LAGE-1a comprises SEQ ID NO: 10 or SEQ ID NO: 58, or an amino acid sequence that has at least 75%, 80%, 85%, 90%, 95%, 96%, 97%, 98%, or 99% identity to SEQ ID NO: 10 or SEQ ID NO: 58. An exemplary murine β-chain constant region is depicted in SEQ ID NO: 51. Accordingly, in certain embodiments, a T-cell receptor that binds to NY-ESO-1 and/or LAGE-1a comprises SEQ ID NO: 51, or an amino acid sequence that has at least 75%, 80%, 85%, 90%, 95%, 96%, 97%, 98%, or 99% identity to SEQ ID NO: 51.

For example, in certain embodiments, a T-cell receptor that binds to NY-ESO-1 and/or LAGE-1a comprises (i) an α-chain that comprises the amino acid sequence of SEQ ID NO: 37 or SEQ ID NO: 84, or an amino acid sequence that has at least 75%, 80%, 85%, 90%, 95%, 96%, 97%, 98%, or 99% identity to SEQ ID NO: 37 or SEQ ID NO: 84, and/or (ii) a β-chain that comprises the amino acid sequence of SEQ ID NO: 39 or SEQ ID NO: 86, or an amino acid sequence that has at least 75%, 80%, 85%, 90%, 95%, 96%, 97%, 98%, or 99% identity to SEQ ID NO: 39 or SEQ ID NO: 86.

In certain embodiments, a T-cell receptor that binds to NY-ESO-1 and/or LAGE-1a comprises (i) an α-chain that comprises the amino acid sequence of SEQ ID NO: 71, or an amino acid sequence that has at least 75%, 80%, 85%, 90%, 95%, 96%, 97%, 98%, or 99% identity to SEQ ID NO: 71, and/or (ii) a β-chain that comprises the amino acid sequence of SEQ ID NO: 73, or an amino acid sequence that has at least 75%, 80%, 85%, 90%, 95%, 96%, 97%, 98%, or 99% identity to SEQ ID NO: 73.

T-cell receptors of the invention typically are glycosylated when expressed in transfected T-cells. In one aspect of the invention, the glycosylation pattern, for example, the N-glycosylation pattern of transfected T-cell receptors may be modified through mutagenesis wherein one or more of the N-glycosylation sites of a T-cell receptor, such as constant region glycosylation sites, are removed. Such mutations include, for example, a change in the key glycosylation site NXS/T to QXS/T in the constant region of the α- and/or β-chains. The T-cell receptor could possess the NXS/T to QXS/T mutation in one of the α- or β-chains or in both chains. Alternatively, in a chimeric protein, for example, a specific human/mouse chimera protein described herein, the key glycosylation site NQT in the murine constant region can be modified to QQT. In another aspect of the invention, cysteine residues may be engineered into the receptor protein enabling the formation of inter-chain disulfide bonds which can stabilize, for example, the resulting refolded soluble T-cell receptors. Furthermore, alanine residues in a T-cell receptor CDR3 region can be substituted with alternative amino acid residues to modulate the binding characteristics of the receptor. Mutagenesis can be carried out with a panel of primers carrying various mutations, followed by performance of functional assays as described above. Once a mutation is identified with the desired phenotype, the construct can be sequenced to identify the location of the mutation.

For example, in certain embodiments, a T-cell receptor that binds to NY-ESO-1 and/or LAGE-1a comprises (i) an α-chain that comprises the amino acid sequence of SEQ ID NO: 75, or an amino acid sequence that has at least 75%, 80%, 85%, 90%, 95%, 96%, 97%, 98%, or 99% identity to SEQ ID NO: 75, and/or (ii) a β-chain that comprises the amino acid sequence of SEQ ID NO: 77 or SEQ ID NO: 97, or an amino acid sequence that has at least 75%, 80%, 85%, 90%, 95%, 96%, 97%, 98%, or 99% identity to SEQ ID NO: 77 or SEQ ID NO: 97.

In certain embodiments, a T-cell receptor that binds to NY-ESO-1 and/or LAGE-1a comprises (i) an α-chain that comprises the amino acid sequence of SEQ ID NO: 79, or an amino acid sequence that has at least 75%, 80%, 85%, 90%, 95%, 96%, 97%, 98%, or 99% identity to SEQ ID NO: 79, and/or (ii) a β-chain that comprises the amino acid sequence of SEQ ID NO: 81, or an amino acid sequence that has at least 75%, 80%, 85%, 90%, 95%, 96%, 97%, 98%, or 99% identity to SEQ ID NO: 81.

In certain embodiments, a T-cell receptor that binds to NY-ESO-1 and/or LAGE-1a comprises (i) an α-chain that comprises the amino acid sequence of SEQ ID NO: 31 or SEQ ID NO: 33, or an amino acid sequence that has at least 75%, 80%, 85%, 90%, 95%, 96%, 97%, 98%, or 99% identity to SEQ ID NO: 31 or SEQ ID NO: 33, and/or (ii) a β-chain that comprises the amino acid sequence of SEQ ID NO: 35, or an amino acid sequence that has at least 75%, 80%, 85%, 90%, 95%, 96%, 97%, 98%, or 99% identity to SEQ ID NO: 35.

In certain embodiments, a T-cell receptor that binds to NY-ESO-1 and/or LAGE-1a comprises (i) an α-chain that comprises the amino acid sequence of SEQ ID NO: 41, or an amino acid sequence that has at least 75%, 80%, 85%, 90%, 95%, 96%, 97%, 98%, or 99% identity to SEQ ID NO: 41, and/or (ii) a β-chain that comprises the amino acid sequence of SEQ ID NO: 73, or an amino acid sequence that has at least 75%, 80%, 85%, 90%, 95%, 96%, 97%, 98%, or 99% identity to SEQ ID NO: 73.

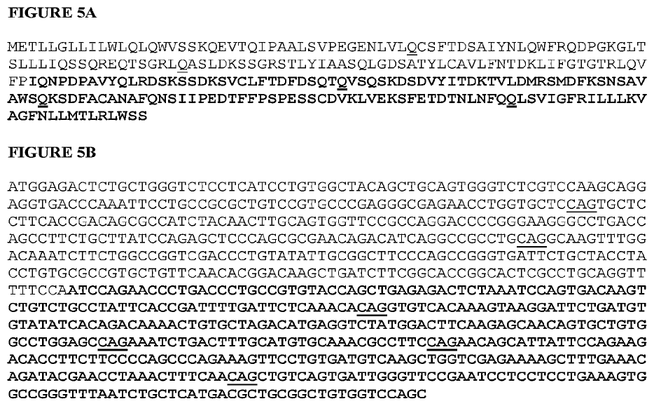

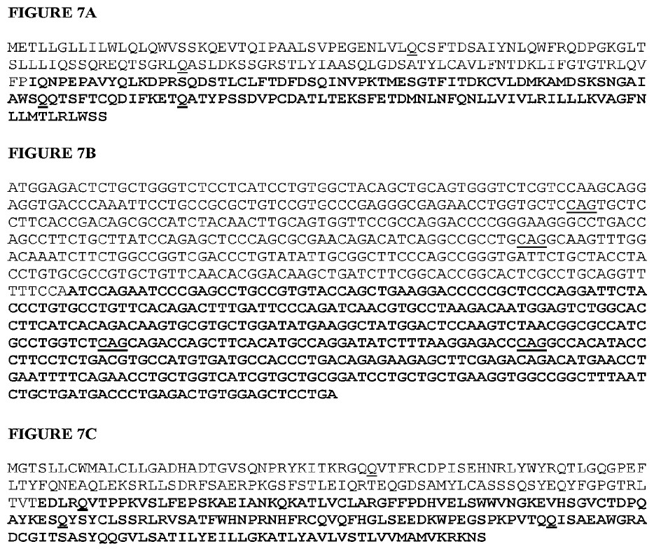

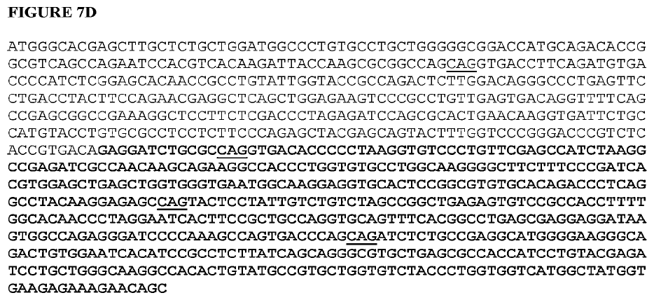

In certain embodiments, a T-cell receptor that binds to NY-ESO-1 and/or LAGE-1a comprises one or more mutations (e.g., NXS/T to QXS/T mutations) depicted in FIG. 5 or FIG. 7. For example, in certain embodiments, a T-cell receptor that binds to NY-ESO-1 and/or LAGE-1a comprises a substitution of an asparagine to glutamine at (i) a position corresponding to position 41, 82, 162, 196 and/or 243 of SEQ ID NO: 75, (ii) a position corresponding to position 37 and/or 200 of SEQ ID NO: 77, (iii) a position corresponding to position 37 and/or 201 of SEQ ID NO: 97, (iv) a position corresponding to position 41, 82, 196, and/or 210 of SEQ ID NO: 79, and/or (v) a position corresponding to position 37, 136, 198, and/or 247 of SEQ ID NO: 81.

In addition, the invention provides soluble versions of the T-cell receptors. Such soluble receptors may be engineered by removing of any portion of the intracellular or transmembrane domains of the TCR α-chain and/or β-chain. See, for example, U.S. Pat. No. 8,519,100, which describes the synthesis of soluble T-cell receptors. Soluble T-cell receptors may comprise the extracellular portion of the TCR as two individual soluble proteins, or as one single chain molecule linked by methods known in the art (Walseng et al. PLOS ONE (2015) 10:1371).

The binding characteristics of the T-cell receptors and T-cell receptor constructs herein can be determined using approaches known in the art. For example, binding affinity (inversely proportional to the equilibrium constant K_{D}) and binding half-life (expressed as T_{1/2}) can be determined by any appropriate method. It is contemplated that doubling the affinity of a T-cell receptor results in halving the K_{D}. T_{1/2} is calculated as ln 2 divided by the off-rate (k_{off}). As a result, doubling of T_{1/2} results in a halving of k_{off}, and K_{D} values. K_{off} values for T-cell receptors often are measured for soluble forms of the receptors, i.e. those forms which are truncated to remove hydrophobic transmembrane domain and the intracellular domain. It is to be understood that a given T-cell receptor satisfies the requirement it has a binding affinity for, and/or a binding half-life for, the SLLMWITQC (SEQ ID NO: 1)-HLA-A2 and/or SLLMWITQCFL (SEQ ID NO: 28)-HLA-A2 complex if a soluble form of that T-cell receptor analog lacking the transmembrane and intracellular domains meets that requirement. Preferably the binding affinity or binding half-life of a given T-cell receptor or T-cell receptor construct is measured several times, for example, 3 or more times, using the same assay protocol, and an average of the results is taken. In certain embodiments these measurements are made using Surface Plasmon Resonance (BIAcore).

For example, a T-cell receptor of the invention may have a K_{D} for the SLLMWITQC (SEQ ID NO: 1)-HLA-A2 and/or SLLMWITQCFL (SEQ ID NO: 28)-HLA-A2 complex of 8 μM or lower, 5 μM or lower, 1 μM or lower, 500 nM or lower, 400 nM or lower, 300 nM or lower, 200 nM or lower, 175 nM or lower, 150 nM or lower, 125 nM or lower, 100 nM or lower, 75 nM or lower, 50 nM or lower, 25 nM or lower, 10 nM or lower, 1 nM or lower, or 0.1 nM or lower.

In certain embodiments, a T-cell receptor of the invention may have a K_{D} for the SLLMWITQC (SEQ ID NO: 1)-HLA-A2 and/or SLLMWITQCFL (SEQ ID NO: 28)-HLA-A2 complex of from about 5 μM to about 0.1 nM, from about 5 μM to about 1 nM, from about 5 μM to about 10 nM, from about 5 μM to about 100 nM, from about 5 μM to about 500 nM, from about 5 μM to about 1 μM or lower, from about 1 μM to about 0.1 nM, from about 1 μM to about 1 nM, from about 1 μM to about 10 nM, from about 1 μM to about 100 nM, from about 1 μM to about 500 nM, from about 500 nM to about 0.1 nM, from about 500 nM to about 1 nM, from about 500 nM to about 10 nM, from about 500 nM to about 100 nM, from about 100 nM to about 0.1 nM, from about 100 nM to about 1 nM, from about 100 nM to about 10 nM, from about 10 nM to about 0.1 nM, from about 10 nM to about 1 nM, or from about 1 nM to about 0.1 nM.

In certain embodiments, a T-cell receptor of the invention, when expressed as a soluble form of the T-cell receptor lacking the transmembrane and intracellular domains, may have a K_{D} for the SLLMWITQC (SEQ ID NO: 1)-HLA-A2 and/or SLLMWITQCFL (SEQ ID NO: 28)-HLA-A2 complex of 8 μM or lower, 5 μM or lower, 1 μM or lower, 500 nM or lower, 400 nM or lower, 300 nM or lower, 200 nM or lower, 175 nM or lower, 150 nM or lower, 125 nM or lower, 100 nM or lower, 75 nM or lower, 50 nM or lower, 25 nM or lower, 10 nM or lower, 1 nM or lower, or 0.1 nM or lower.

In certain embodiments, a T-cell receptor of the invention may have a K_{D} for the SLLMWITQC (SEQ ID NO: 1)-HLA-A2 and/or SLLMWITQCFL (SEQ ID NO: 28)-HLA-A2 complex that is lower than the K_{D} of a reference T-cell receptor (e.g., 1G4LY TCR or 1G4 TCR as described in Example 7 herein) for the SLLMWITQC (SEQ ID NO: 1)-HLA-A2 and/or SLLMWITQCFL (SEQ ID NO: 28)-HLA-A2 complex. For example the T-cell receptor of the invention may have a K_{D} that is at least 1 μM, 500 nM, 400 nM, 300 nM, 200 nM, 175 nM, 150 nM, 125 nM, 100 nM, 75 nM, 50 nM, 25 nM, 10 nM, 1 nM, or 0.1 nM lower than the K_{D} of the reference T-cell receptor. K_{D} may be measured by any method known in the art, for example, surface plasmon resonance as described in Example 7 herein.

T-cell receptors of the invention may have a binding half-life (T_{1/2}) for the complex of ≥1.5 s, ≥3 s, ≥10 s, ≥20 s, ≥40 s, ≥60 s, ≥600 s, or ≥6000 s. The kₒₙ may be ≥10³M⁻¹S⁻¹, ≥10⁴M⁻¹S⁻¹, ≥10⁵M⁻¹S⁻¹, ≥10⁶M⁻¹S⁻¹, or ≥10⁷M⁻¹S⁻¹ and/or the k_{off} may be ≤10⁻¹S⁻¹, ≤10⁻²S⁻¹, ≤10⁻³S⁻¹, ≤10⁻⁴S⁻¹, ≤10⁻⁵S⁻¹, or ≤10⁻⁶S⁻¹.

The invention further provides bispecific T-cell receptor proteins comprising a T-cell receptor of the invention, as described above, including single chain T-cell receptors, in association with an additional binding moiety (for example, an antibody, or a different T-cell receptor, or an antigen binding fragment of any of the foregoing) that binds a second antigen other than the NY-ESO-1 and/or LAGE-1a antigen (Garber (1994) NAT. REV. DRUG DISCOV. 13:799-801). For example, the bispecific receptor protein may be a fusion protein comprising a T-cell receptor of the invention fused to an immune-modulating polypeptide such as an antibody or an antigen binding fragment thereof.

In one embodiment, the bispecific T-cell protein comprises a T-cell receptor of the invention, including single chain T-cell receptors, or fragments thereof, associated with an antibody or antigen binding fragment thereof, that binds the CD3 antigen. In an embodiment, the bispecific antibody may be a fusion protein comprising a linker sequence (for example, an amino acid linker sequence 5, 10, 15, 20, 25, 30, 35, 40, 45 or 50 amino acids in length) that links the T-cell receptor polypeptide to the anti-CD3 binding moiety. In one embodiment, an anti-CD3 binding moiety, for example, a single-chain variable region (sFv) is fused, via an amino acid linker, to the N-terminus of a T-cell receptor β-chain. Under certain circumstances it can be desirable to remove the transmembrane domain and/or the intracellular domains from the constant region of the α-chain and/or the β-chain when creating the fusion constructs. For example, the invention also provides soluble versions of the T-cell receptors, including bispecific T-cell receptors proteins. Such soluble receptors may be engineered by the removal of any portion of the intracellular or transmembrane domains of the TCR α-chain and/or β-chain. Bispecific T-cell proteins that include anti-CD3 polypeptides and methods of making such proteins are described in U.S. Pat. No. 8,519,100, the disclosure of which is incorporated by reference herein. U.S. Pat. No. 8,519,100 describes methods for designing fusion constructs, making expression constructs, transfecting expression vectors into host cells, expressing fusion constructs, harvesting, purifying and refolding the fusion constructs. The expression of such a bispecific fusion T-cell receptor in engineered T-cells (as described in detail below) is designed to enhance the reactivity and cytotoxicity of the T-cells towards targeted cancer cells or tumor cells of a subject. Both bispecific T-cell receptors and soluble T-cell receptors allow the TCR or the peptide/MHC complex binding motif of the TCR to be utilized without the need of autologous and/or allogeneic cell transduction.

In another embodiment of the invention, chimeric T-cell receptors are provided wherein a T-cell receptor of the invention is expressed as a fusion with a second polypeptide. Such second polypeptides include for example, cytotoxic agents such as ricin, diphtheria toxin, bacterial exotoxin A, DNase and RNase. Such chimeric T-cell receptors may be useful in targeting such cytotoxic agents to cancer or tumor cells of a subject.

Also included in the invention is a T-cell receptor of the invention, as well as fragments and functional variants thereof, that are modified to comprise a detectable label. For example, soluble T-cell receptors of the invention maybe associated (covalently or non-covalently associated) with a detectable moiety such as a radioisotope, a fluorophore (e.g., fluorescein isothiocyanate (FITC), phycoerythrin (PE)), an enzyme (e.g., alkaline phosphatase, horseradish peroxidase), or a particle (e.g., a gold particle). Such molecules can be used in diagnostic screens to detect the presence of cancer cells within a subject.

Alternatively, an effector therapeutic agent may be associated with a T-cell receptor of the invention, as well as fragments and functional variants thereof. Such agents include for example, immune modulating antibody fragments such as anti-CD3 or anti-CD16 antibody fragments, toxins, radioisotopes, immuno-stimulants such as IL-2, IFN-γ, CCL21, or GM-CSF, chemotherapeutic agents or a drug. Such T-cell receptors can be used to target delivery of the effector molecule to cancer cells of a subject.

### III. Nucleic Acids Encoding T-Cell Receptors and Engineered Variants Thereof

The invention further provides nucleic acids encoding the T-cell receptors of the invention (including non-naturally occurring T-cell receptors) as well as fragments and functional variants thereof.

The invention encompasses nucleic acids that encode the T-cell receptors of the invention, variants and fragments of T-cell receptors, fusion proteins such as single chain T-cell receptors, and bispecific receptor proteins. Nucleic acids include, but are not limited to, those sequences encoding full length α- and β-chains, α- and β-chain variable regions as well as α- and β-chain regions containing one or more of the CDR₁₃ regions of SEQ ID NO: 5-7 (α-chain) and SEQ ID NO: 11-13 (β-chain). In an additional aspect, the invention provides an expression vector, for example, a viral expression vector, comprising one or more of a disclosed nucleic acid sequence. In certain embodiments, the viral vector is a lentivirus vector.

In one aspect, the invention provides an isolated, recombinant nucleic acid encoding an α-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). In one aspect, the invention provides an isolated, recombinant nucleic acid encoding an α-chain of a T-cell receptor immunoreactive with an epitope of a NY-ESO-1 and/or LAGE-1a protein comprising the amino acid sequence SLLMWITQC (SEQ ID NO: 1). The nucleic acid comprises one or more of the following nucleotide sequences: (i) a nucleotide sequence encoding an amino acid sequence of SEQ ID NO: 2 or SEQ ID NO: 87, or an amino acid sequence having greater than 97%, 98% or 99% identity to the amino acid sequence of SEQ ID NO: 2 or SEQ ID NO: 87; (ii) a nucleotide sequence encoding an α-chain variable region amino acid sequence of SEQ ID NO: 3 or SEQ ID NO: 83, or an amino acid sequence having greater than 96%, 97%, 98% or 99% identity to the amino acid sequence of SEQ ID NO: 3 or SEQ ID NO: 83; (iii) a nucleotide sequence of SEQ ID NO: 15 or SEQ ID NO: 100; (iv) a nucleotide sequence encoding an α-chain CDR₃ amino acid sequence of SEQ ID NO: 7; (iv) a nucleotide sequence encoding an α-chain CDR₁ amino acid sequence of SEQ ID NO: 5; and (vi) a nucleotide sequence encoding an α-chain CDR₂ amino acid sequence of SEQ ID NO: 6.

In another aspect, the invention provides an isolated, recombinant nucleic acid encoding a T-cell receptor β-chain immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28). In one aspect, the invention provides an isolated, recombinant nucleic acid encoding a β-chain of a T-cell receptor immunoreactive with an epitope of a NY-ESO-1 and/or LAGE-1a protein comprising the amino acid sequence SLLMWITQC (SEQ ID NO: 1). The nucleic acid comprises one or more of the following nucleotide sequences: (i) a nucleotide sequence encoding an amino acid sequence of SEQ ID NO: 8, SEQ ID NO: 88, SEQ ID NO: 95, or SEQ ID NO: 99, or an amino acid sequence having greater than 97%, 98% or 99% identity to the amino acid sequence of SEQ ID NO: 8, SEQ ID NO: 88, SEQ ID NO: 95, or SEQ ID NO: 99; (ii) a nucleotide sequence encoding a β-chain variable region amino acid sequence of SEQ ID NO: 9 or SEQ ID NO: 85, or an amino acid sequence having greater than 97%, 98% or 99% identity to the amino acid sequence of SEQ ID NO: 9 or SEQ ID NO: 85; (iii) a nucleotide sequence of SEQ ID NO: 21, SEQ ID NO: 96, or SEQ ID NO: 102; (iv) a nucleotide sequence encoding a β-chain CDR₃ amino acid sequence of SEQ ID NO: 13; (v) a nucleotide sequence encoding a β-chain CDR₁ amino acid sequence of SEQ ID NO: 11; and (vi) a nucleotide sequence encoding a β-chain CDR₂ amino acid sequence of SEQ ID NO: 12.

In another aspect, the invention provides an isolated, recombinant nucleic acid encoding a T-cell receptor immunoreactive with an epitope of an NY-ESO-1 and/or LAGE-1a protein comprising the amino acid sequence SLLMWITQC (SEQ ID NO: 1) and/or SLLMWITQCFL (SEQ ID NO: 28). In one aspect, the invention provides an isolated, recombinant nucleic acid encoding a T-cell receptor immunoreactive with an epitope of a NY-ESO-1 and/or LAGE-1a protein comprising the amino acid sequence SLLMWITQC (SEQ ID NO: 1). The T-cell receptor comprises an α-chain and a β-chain each comprising a CDR₁, CDR₂, and a CDR₃, wherein the α-chain CDR₃ comprises the amino acid sequence of SEQ ID NO: 7, and the β-chain CDR₃ comprises the amino acid sequence of SEQ ID NO: 13. Optionally, or in addition, the α-chain CDR₁ comprises the amino acid sequence of SEQ ID NO: 5, and the β-chain CDR₁ comprises the amino acid sequence of SEQ ID NO: 11. Optionally, or in addition, the α-chain CDR₂ comprises the amino acid sequence of SEQ ID NO: 6, and the β-chain CDR₂ comprises the amino acid sequence of SEQ ID NO: 12.

It is contemplated that, for each of the nucleic acids described herein, the nucleic acids may encode an amino acid sequence having at least 1, 2, 3, 4, 5, 6, 7, 8, 9, 10, 11, 12, 13, 14, 15, 16, 17, 18, 19, 20 amino acid residues not present at a given position in T-cell receptor. For example, contemplated herein are nucleic acids encoding amino acid sequences having at least 1, 2, 3, 4, 5, 6, 7, 8, 9, 10, 15, or 20 amino acid substitutions not present in SEQ ID NOs: 2, 3, 4, 8, 9, 10, 83, 85, 87, 88, 95, or 99, and/or include codon optimization to enhance the expression of the α-chain and/or the β-chain of the T-cell receptor in a given cell type or subject.

For each of the foregoing aspects, the nucleic acid may encode a single chain T-cell receptor, optionally where the α-chain is linked to the β-chain via an amino acid linker. In an embodiment of the invention, the nucleic acids can encode a single chain T-cell receptor, or a bispecific T-cell receptor fusion protein. In a specific embodiment, the nucleic acid encodes an antibody that is an immune-modulating antibody.

In another aspect, the invention provides an isolated, recombinant nucleic acid encoding a fusion protein comprising a T-cell receptor immunoreactive with an epitope of an NY-ESO-1 and/or LAGE-1a protein comprising the amino acid sequence SLLMWITQC (SEQ ID NO: 1) and/or SLLMWITQCFL (SEQ ID NO: 28) and a CD34 protein or a truncated form of a CD34 protein. In certain embodiments, the truncated CD34 protein lacks an intracellular signaling domain. For example, in one embodiment, the invention provides an isolated, recombinant nucleic acid encoding the amino acid sequence of SEQ ID NO: 29 or comprising the nucleotide sequence of SEQ ID NO: 30.

The nucleic acids may be recombinant nucleic acids. The nucleic acids may be produced via chemical synthesis on a synthesizer and/or via enzymatic ligation reactions using procedures known in the art. See, for example, Sambrook et al., Molecular Cloning: A Laboratory Manual, 3.sup.rd ed., Cold Spring Harbor Press, Cold Spring Harbor, N.Y. 2001; and Ausubel et al., Current Protocols in Molecular Biology, Greene Publishing Associates and John Wiley & Sons, NY, 1994). The nucleic acids encoding the T-cell receptors of the invention may be isolated using a variety of different methods known to those skilled in the art. For example, a cDNA library constructed using RNA from cells or tissue known to express a T-cell receptor, i.e., T-cells, can be screened using a labeled T-cell receptor nucleic acid probe. Alternatively, a genomic library may be screened to derive nucleic acid molecules encoding a T-cell receptor. Further, T-cell receptor nucleic acid sequences may be derived by performing PCR using oligonucleotide primers designed on the basis of the T-cell receptor nucleotide sequences disclosed herein. The template for the reaction may be cDNA obtained by reverse transcription of mRNA prepared from cell lines or tissue known to express the T-cell receptor.

The nucleic acid can comprise any nucleotide sequence which encodes any of the T-cell receptors of the invention, as well as fragments and functional variants thereof, described herein. The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleic acids of the invention include those nucleic acids (i) that hybridize to the nucleotide sequences encoding the T-cell receptors of the invention described herein under stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I Green Publishing Associates, Inc., and John Wiley & sons, Inc., New York, at p. 2.10.3); or (ii) that hybridize under less stringent conditions, such as moderately stringent conditions, e.g., washing in 0.2×SSC/0.1% SDS at 42° C. (Ausubel et al., 1989 supra).

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. Sequence identity can be determined as discussed above.

In certain embodiments, nucleic acids of the invention include those nucleic acids as described above, wherein the codon usage of the nucleic acid has been optimized to enhance the expression of the α-chain and/or the β-chain of a T-cell receptor in a particular cell type or subject. Nucleic acid sequences may be codon optimized to improve stability or heterologous expression in host cells without changing the encoded amino acid sequence. For example, codon optimization may be used to remove sequences that negatively impact gene expression, transcript stability, protein expression or protein stability, such as transcription splice sites, DNA instability motifs, polyadenylation sites, secondary structure, AU-rich RNA elements, secondary open reading frames (ORFs), codon tandem repeats, or long range repeats. Codon optimization may also be used to adjust the G/C content of a sequence of interest. Codon optimization replaces codons present in a DNA sequence with preferred codons encoding the same amino acid, for example, codons preferred for mammalian expression. Thus, the amino acid sequence is not altered during the process. Codon optimization can be performed using gene optimization software. The codon optimized nucleotide sequence is translated and aligned to the original protein sequence to ensure that no changes were made to the amino acid sequence. Methods of codon optimization are known in the art and are described, for example, in U.S. Application Publication No. 2008/0194511 and U.S. Pat. No. 6,114,148.

The invention also encompasses recombinant expression vectors that contain any of the nucleic acids described herein. Accordingly, the present invention encompasses a recombinant expression vector comprising a nucleic acid encoding a T-cell receptor described herein. The vector may comprise nucleic acids that encode the T-cell receptors described herein, variants and fragments of the T-cell receptors, fusion proteins such as single chain T-cell receptors, and bispecific receptor proteins. Nucleic acids include, but are not limited to, those sequences encoding the full length α- and β-chains, the α- and β-chain variable regions as well as α- and β-chain regions containing one or more of the CDR1-3 regions of SEQ ID NOs: 5-7 (α-chain) and SEQ ID NOs: 11-13 (β-chain).

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides.

It is contemplated that a variety of recombinant expression vectors can be used to express the T-cell receptors and T-cell receptor constructs described herein, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as kGT10, kGT11, kZapII (Stratagene), kEMBL4, and kNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech). Preferably, the recombinant expression vector is a viral vector, e.g., a retroviral vector or a lentiviral vector.

The recombinant expression vectors of the invention can be prepared using standard recombinant DNA techniques. (Ausubel et al. (1989) supra.; Sambrook et al. (2001) supra.). Constructs of expression vectors, which are circular or linear, can be prepared to contain a replication system functional in a prokaryotic or eukaryotic host cell. Replication systems can be derived, for example, from ColE1, 2μ plasmid, λ, SV40, bovine papilloma virus, and the like.

In one embodiment, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or non-native promoter operably linked to the nucleotide sequence encoding a T-cell receptor, polypeptide, or protein (including functional variants thereof), or to the nucleotide sequence which is complementary to or which hybridizes to the nucleotide sequence encoding a T-cell receptor, polypeptide, or protein (including functional variants thereof). It is contemplated that the selection of appropriate promoters, for example, strong, weak, inducible, tissue-specific and developmental-specific, is within the ordinary skill of the artisan. Similarly, it is contemplated that the combination of a nucleotide sequence with a promoter is also within the skill of the artisan. The promoter can be a non-viral promoter or a viral promoter, e.g., murine stem cell virus (MSCV), a cytomegalovirus (CMV) promoter, an SV40 promoter, an RSV promoter, and a promoter found in the long-terminal repeat of the murine stem cell virus.

The recombinant expression vectors can be designed for transient expression, stable expression, or for both. Also, the recombinant expression vectors can be made for constitutive expression or for inducible expression. Further, the recombinant expression vectors can be made to include a suicide gene.

In one embodiment, viral based vector systems can be used to express the T-cell receptors and/or engineered T-cell receptor constructs. Such viral vector based systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene.

### IV. Genetically Engineered Host Cells

In addition, the invention provides genetically engineered host cells comprising any of the recombinant nucleic acids and/or expression vectors described hereinabove. The host cell can be a eukaryotic cell, e.g., animal (for example, human), fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, progenitor cells such as hematopoietic or progenitor stem cells and the like. For purposes of amplifying or replicating the recombinant expression vector, the host cell can be a prokaryotic cell, e.g., a DH5α cell.

For purposes of producing a recombinant T-cell receptor, polypeptide, or protein, the host cell preferably is a mammalian cell, for example, a human cell. While the host cell can be of any cell type, can originate from any type of tissue, and can be of any developmental stage, the host cell can be a peripheral blood lymphocyte (PBL) or a peripheral blood mononuclear cell (PBMC), or a Natural Killer (NK) cell. More preferably, the host cell is a T-cell. The T-cell can be any T-cell, such as a cultured T-cell, e.g., a primary T-cell, or a T-cell cell from a cultured T-cell line, e.g., Jurkat, SupTi, etc., or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. Preferably, the T-cell is a human T-cell, which can be an autologous or heterologous cell. The T-cell can be any type of T-cell and can be of any developmental stage, including but not limited to, CD4⁺/CD8⁺ double positive T-cells, CD4⁺ helper T-cells, e.g., Th₁ and Th₂ cells, CD4⁺ T-cells, CD8⁺ T-cells (e.g., cytotoxic T-cells), tumor infiltrating lymphocytes (TILs), memory T-cells (e.g., central memory T-cells and effector memory T-cells), naive T-cells, and the like. The T cells can also be previously engineered to knockdown or delete molecules that elicit immune rejections when otherwise transferred to an allogeneic host.

The cells can include autologous cells derived from a subject to be treated, or alternatively allogenic cells derived from a donor.

The population of cells can be a heterogeneous population comprising the host cell comprising any of the recombinant expression vectors described herein, in addition to at least one other cell, e.g., a host cell (e.g., a T-cell), which does not comprise any of the recombinant expression vectors, or a cell other than a T-cell, e.g., a B-cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

It is contemplated that standard transfection methods can be used to produce bacterial, mammalian, yeast or insect cell lines that express large quantities of protein, which are then purified using standard techniques (see, e.g., Colley et al. (1989) J. BIOL. CHEM. 264:17619-17622; Deutscher, ed. (1990) Guide to Protein Purification, METHODS IN ENZYMOLOGY, vol. 182).

Conventional gene transfer methods can be used to introduce nucleic acids encoding the T-cell receptors and T-cell receptor constructs described herein into host cells and target tissues. In certain embodiments, the nucleic acids encoding the T-cell receptors and T-cell receptor constructs are introduced into host cells for in vivo or ex vivo gene therapy uses. Methods of nucleic acid delivery include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Lipofection is described in e.g., U.S. Pat. Nos. 5,049,386, 4,946,787, and 4,897,355 and lipofection reagents are sold commercially (e.g., Transfectam and Lipofectin). The preparation of lipid:nucleic acid complexes, including targeted liposomes such as immunolipid complexes, is well known to one of skill in the art (see, e.g., Crystal (1995) SCIENCE 270:404-410; Blaese et al. (1995) CANCER GENE THER. 2:291-297; Behr et al. (1994) BIOCONJUGATE CHEM. 5:382-389; Remy et al. (1994) BIOCONJUGATE CHEM. 5:647-654; Gao et al. (1995) GENE THER. 2:710-722; Ahmad et al. (1992) CANCER RES. 52:4817-4820; U.S. Pat. Nos. 4,186,183; 4,217,344; 4,235,871; 4,261,975; 4,485,054; 4,501,728; 4,774,085; 4,837,028; and 4,946,787).

Viral vector delivery systems, such as of RNA or DNA viral based systems, may also be used to introduce nucleic acids encoding the T-cell receptors and T-cell receptor constructs into host cells. Viral vectors can be administered directly to patients (in vivo) or they can be used to treat cells in vitro (ex vivo) and the modified cells then are administered to a subject. Conventional viral based systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene.

### V. T-Cell Receptor Mediated Treatments

It is contemplated that pharmaceutical compositions (as described herein), comprising T-cell receptors, including fragments and functional variants thereof, nucleic acids, recombinant expression vectors, host cells, or populations of cells can be used in methods of treating or preventing cancer. The T-cell receptors, and functional variants thereof, are believed to bind specifically to the NY-ESO-1 and/or LAGE-1a antigen, such that the T-cell receptor, or related polypeptides or functional variants thereof, when expressed by a cell is able to mediate binding to a cell expressing NY-ESO-1 and/or LAGE-1a.

In this regard, the invention provides a method of treating or preventing cancer in a subject, comprising administering to the subject any of the pharmaceutical compositions, T-cell receptors (and functional variants thereof), polypeptides, or proteins described herein, any nucleic acid or recombinant expression vector comprising a nucleotide sequence encoding any of the T-cell receptors (and functional variants thereof), polypeptides, proteins described herein, or any host cell or population of cells comprising a recombinant vector which encodes any of the T-cell receptors (and functional variants thereof), polypeptides, or proteins described herein, in an amount effective to treat or prevent cancer in the subject.

The terms “treating”, “treatment” or “prevention” of a disease (or a condition or a disorder) as used herein refers to preventing the disease from occurring in a subject that may be predisposed to the disease but does not yet experience or exhibit symptoms of the disease (preventing), inhibiting the disease (slowing or arresting its development), providing relief from the symptoms or side-effects of the disease (including palliative treatment), and relieving the disease (causing regression of the disease). With regard to cancer, these terms also mean that the life expectancy of an individual affected with a cancer may be increased or that one or more of the symptoms of the disease is reduced. Compositions can be formulated by any of the means known in the art.

Representative cancers to be treated include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, vulvar tumor, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, liver cancer, medulloblastoma, neuroblastoma, brain tumors generally, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, esophageal cancer, hairy cell leukemia, kidney cancer, multiple myeloma, oral cancer, pancreatic cancer, primary central nervous system lymphoma, skin cancer, small-cell lung cancer, among others. Some of these cancers naturally express NY-ESO-1 and/or LAGE-1, while others can be induced to express NY-ESO-1 and/or LAGE-1a through the use of HDAC inhibitors.

As described herein, in certain embodiments, the T-cell receptors described herein recognize the NY-ESO-1 and/or LAGE-1a antigen in HLA A2 restrictive manner. Given the high percentage of HLA-A2 restricted subjects in North American (40%) and East Asian (30%) populations, the compositions and methods of the invention may be particularly well suited for treatment of cancer in this population subset.

In a specific embodiment, the T-cell receptors and T-cell receptor constructs may be used in adoptive T-cell immunotherapy for treatment of cancer. In such treatments, T-cells are genetically engineered to express a T-cell receptor or T-cell receptor constructs described herein followed by introduction of the engineered cells into the subject. Such engineered T-cells, by virtue of their T-cell receptor expression, are targeted to cancer cells expressing the NY-ESO-1 and/or LAGE-1a antigen. Preferably, such T-cells are cells into which a nucleic acid encoding the α-chain and/or β-chain, or fragments and functional variants thereof, of a T-cell receptor have been introduced. T-cells for use in the adoptive immunotherapy include autologous cells derived from a subject to be treated or allogenic cells derived from a donor who is an acceptable HLA-match.

When such a cancer therapy is performed, T-cell receptor encoding nucleic acids can be introduced into peripheral blood lymphocytes obtained from the cancer subject to be treated. Prior to reintroduction into the cancer subject, the lymphocytes into which T-cell receptor encoding nucleic acid has been introduced, as described above, may be cultured ex vivo to obtain a large amount of NY-ESO-1 and/or LAGE-1a specific lymphocytes. Further, specific subsets of the lymphocytes into which T-cell receptor encoding nucleic acid may be purified, or isolated using methods know to those of skilled in the art. For example, subsets of CD8⁺ T-cells may be purified from the mixed population of peripheral blood lymphocytes, for example, using antibodies against a TCR or regions of a TCR (such as v007-09 or v005-01) expressed on cell surface. Subsets of CD8⁺ T-cells may be purified from the mixed population of peripheral blood lymphocytes also, for example, by coexpressing a TCR with a transgene, such as CD34 or a truncated form CD34, followed by identification of cells using anti-CD34 antibody based methods, for example, flow cytometry or immuno-magnetic methods.

In addition, the adoptive immunotherapy as described above may be combined with other cancer treatments including chemotherapy, radiotherapy and surgery. In a specific embodiment of the invention, the adoptive immunotherapy may be combined with the use of immunological checkpoint inhibitors. Such inhibitors include for example, anti-cytotoxic T-lymphocyte-associated antigen (CTLA-4) antibodies and anti-programmed cell death (PD)-1/PD-ligand-1 (PD-L1) antibodies.

In another embodiment, T-cell receptors, as well as fragments and functional variants thereof, associated an effector therapeutic agent may be used to treat cancer. Such agents include for example, immune modulating antibody fragments such as anti-CD3 or anti-CD16 antibody fragments, toxins, radioisotopes, immuno-stimulants such as IL-2 and IFN-γ, chemotherapeutic agents or a drug. Such T-cell receptors can be used to target delivery of the effector molecule to cancer cells of a subject thereby targeting destruction of the cancer cells.

### VI. Pharmaceutical Compositions and Methods of Administration

For administration to patients, a T-cell receptor of the invention, including functional fragments and variants thereof, nucleic acids, recombinant expression vectors and host cells can be formulated into a pharmaceutical composition with a pharmaceutically acceptable carrier. Pharmaceutical compositions suitable for use in the present invention include compositions wherein the T-cell receptor related compositions are present in an amount effective to achieve the intended purpose. Determination of the effective amounts is within the level of those skilled in the art, especially in light of the detailed disclosure provided herein.

With respect to the pharmaceutical compositions, the carrier can be any of those conventionally used for the particular T-cell receptor related pharmaceutical composition to be administered. Such pharmaceutically acceptable carriers are well-known to those skilled in the art and are readily available. It is preferred that the pharmaceutically acceptable carrier be one which has no detrimental side effects or toxicity under the conditions of use.

The pharmaceutical composition may be in any suitable form, depending upon the desired mode of administration to a patient. The pharmaceutical composition may be adapted for administration by any appropriate route, preferably a parenteral (including subcutaneous, intramuscular, or preferably intravenous) route. The pharmaceutical compositions of the present disclosure may be manufactured in a manner that is itself known, e.g., by means of conventional mixing, dissolving, granulating, dragee-making, levitating, emulsifying, encapsulating, entrapping or lyophilizing processes. Such compositions may be prepared by any method known in the art of pharmacy, for example by mixing the active ingredient with the carrier(s) or excipient(s) under sterile conditions.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Preferably, the pharmaceutical composition is administered by injection, e.g., intravenously. When the composition is a host cell expressing a T-cell receptor of the invention, or a fragment or functional variant thereof, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The amount or dose (e.g., numbers of cells when the composition is one or more cells, i.e., a population of cells) of the pharmaceutical composition administered should be sufficient to effect, e.g., a therapeutic or prophylactic response, in the subject or animal over a reasonable time frame. For example, the dose of a T-cell receptor related composition should be sufficient to bind to a cancer antigen, or detect, treat or prevent cancer in a subject. The dose will be determined by the efficacy of the particular pharmaceutical composition and the condition of the subject (e.g., human), as well as the body weight of the subject (e.g., human) to be treated. Assays for determining an administered dose are well known in the art. The cells can typically be prepared as injectables, especially for intravenous and intraperitoneal administration either as liquid solutions or suspensions.

The dose of the composition can also be determined by the existence, nature and extent of any adverse side effects that might accompany the administration of a particular composition. Typically, the attending physician will decide the dosage of the composition with which to treat each individual patient, taking into consideration a variety of factors, such as age, body weight, general health, diet, sex, route of administration, and the severity of the condition being treated. It is contemplated that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods.

### VII. Diagnostic Methods

Also provided by the present invention are diagnostic methods for use in detection of cancer in a mammal. The method comprises contacting a sample of cells or tissue derived from a subject suspected of having cancer with a T-cell receptor associated with a label, thereby forming a complex, and detecting the complex, wherein detection of the complex is indicative of the presence of cancer in the subject.

Samples for analysis in such methods can be any organ, tissue, cell, or cell extract isolated from a subject, such as a sample isolated from a mammal having cancer. For example, a sample can include, without limitation, cells or tissue (e.g., from a biopsy), blood, serum, tissue or fine needle biopsy samples, or any other specimen, or any extract thereof, obtained from a test subject. A sample may also include sections of tissues such as frozen sections taken for histological purposes.

For purposes of the diagnostic method, the contacting can take place in vitro or in vivo with respect to the subject. Detection of the complex can occur through any number of ways known in the art. For instance, a T-cell receptor can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and particles (e.g., gold particles) as described above.

Finally, this invention provides kits for performing the instant diagnostic methods described herein. Each kit comprises a labeled T-cell receptor reagent, suitable solvents and instructions for using the kits. Such T-cell receptor based diagnostic kits and their methods of manufacture and use are well known.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

Practice of the invention will be more fully understood from the foregoing examples, which are presented herein for illustrative purposes only, and should not be construed as limiting the invention in any way.

### Example 1—Identification of 806 T-Cell Receptor

The following example describes the identification of newly created T-cells (e.g., new cells that contain new proteins and nucleic acids encoding such proteins that have not been identified as naturally occurring in nature) that recognize NY-ESO-1 and/or LAGE-1a epitopes in an HLA restricted manner.

Human T-cells that recognize tumor-associated antigens were isolated from peripheral blood lymphocytes using a “reverse immunology” approach (Zeng et al. (2000) J. IMMUNOL. 165:1153-1159; Zeng et al. (2001) PROC. NATL. ACAD. SCI. USA 98: 3964-3969). In this case, an in vitro sensitization procedure was carried out as described using the HLA-A2 restricted NY-ESO-1:157-165 epitope of SEQ ID NO: 1 (Zeng et al. (2000) supra) Briefly, lymphocytes from various donors were plated in a 96-well flat-bottom plate in the presence of 1 μg/mL of the above mentioned peptide. On days 7 and 14, about 1×10⁵ non-irradiated lymphocytes were pulsed with 10 μg/mL peptide and washed twice. Subsequently, IL-2 at a final concentration of 120 units/mL was added to each well. On day 21, the cells were harvested and incubated with presenting cells overnight prior to collection of supernatants. Primary B cells activated with human IL-4 and CD40 ligands (“B cells”) and immortalized, Epstein-Barr virus infected B-cells (“EVB-B”) were used as presenting cells. Release assays were conducted to detect IFN-γ (BioLegend, San Diego, CA) or GM-CSF (eBioscience, San Diego, CA) as a means for determining the specific activity of T-cells and T-cell receptor-transduced target cells. T-cells from wells with high specific activities were pooled, enriched and then expanded using a rapid T-cell expansion method (Riddell et al. (1990) J. IMMUNOL. METHODS 128: 189-201). The HLA types of the donors and antigen presenting cells were determined using a molecular approach performed at LabCorp (West Hills, CA).

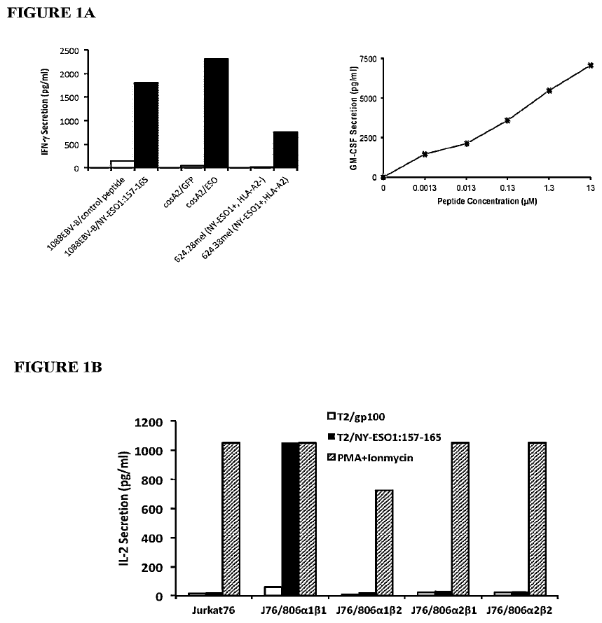

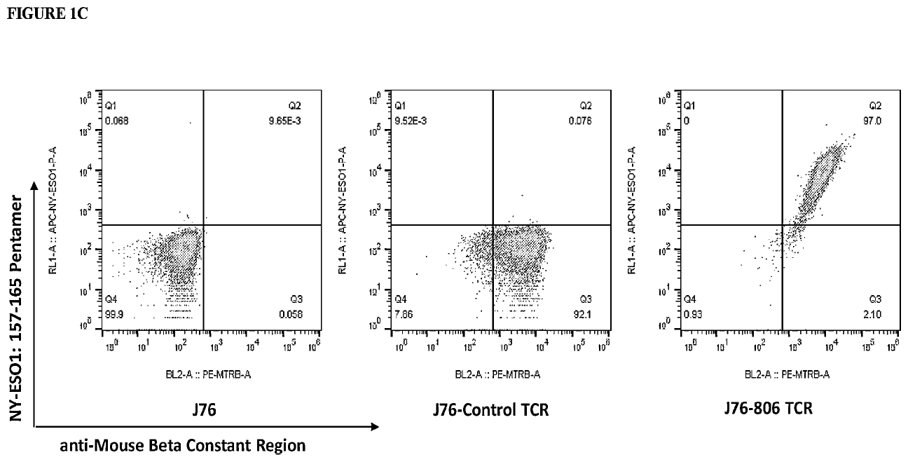

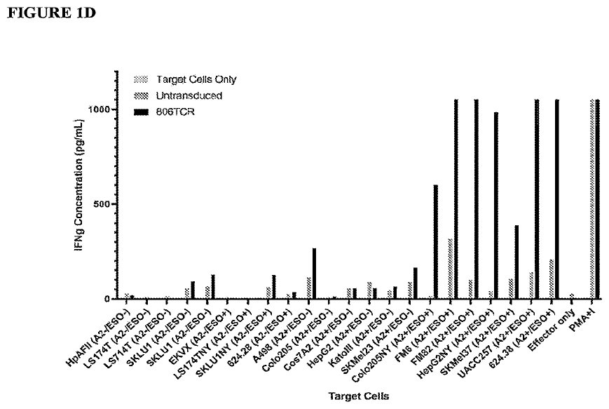

A few wells of cells showed significant growth with specific activities against presenting 1088 B cells pulsed with the NY-ESO-1:157-165 (ESO:157-165) peptide epitope, which has been previously shown to be restricted by HLA-A2 (Jager et al. (1997) J. EXP. MED.). FIG. 1A shows the recognition by the 806 CD8⁺ T-cell line of the ESO:157-165 peptide. The 806 CD8⁺ T-cell line bound the ESO:157-165 peptide when presented by HLA-A2+ 1088-B cells, as determined by an IFN-γ release assay. Similarly, 806 CD8⁺ T-cells recognized NY-ESO-1, but not GFP, presented by cosA2 cells. HLA specificity was confirmed as 806 T-cells recognized the 624.38 melanoma line (HLA-A2+/NY-ESO-1+) which expresses HLA-A2, but not the variant 624.28 melanoma line (HLA-A2−/NY-ESO-1+) which lacks HLA-A2 expression. The 806 CD8⁺ T-cell line was further tested for its ability to recognize the ESO:157-165 peptide epitope at various concentrations by a GM-CSF release assay. Results shown in FIG. 1B suggest a high avidity nature for the TCR peptide interaction. Cells transduced with the 806 α1β1 combination were further tested for binding to HLA-A2/NY-ESO-1:157-165 pentamers. Results, shown in FIG. 1C, demonstrated binding to the NY-ESO-1 pentamer. Additionally, cells transduced with the 806 TCR were assayed for activation upon exposure to a panel of tumor cells. Results, shown in FIG. 1D, demonstrated that transduced cells had specific activity when exposed to A2+/NYESO1+ cells (Colo-205-NYESO1, FM-6, FM-82, HEPG2-NYESO, SK-MEL-37, UACC-257, and MEL-624.38); while they had no specific activity when exposed to A2−/NYESO1− cells (HpAF-II, LS174T, LS714T, and SK-LU-1) or A2+/NYESO1− cells (SK-LU-1-NYESO, MEL-624.28, A549, Colo-205, Cos-7-A2, HepG-2, Kato-III, and SK-MEL-23).

### Example 2—Cloning of 806 T-Cell Receptor

As described in Example 1, the 806 CD8⁺ T-cell line has immunoreactivity with the ESO:157-165 peptide epitope when the peptide is presented by HLA-A2 molecules. The following example describes the cloning and characterization of the T-cell receptor expressed by the 806 CD8⁺ T-cell line that mediates the recognition of ESO:157-165 and in the context of HLA-A2.

The 806 CD8⁺ T-cells identified in Example 1 were subjected to T-cell receptor (TCR) cloning. Total RNA from T-cell cultures (>1×10⁵ cells) was prepared using an RNeasy Mini Kit (Hawthorn, CA). A cDNA library was prepared using a GeneRacer approach (Invitrogen, Carlsbad, CA) that generated a full-length cDNA library with oligo dT and a universal 5′ rapid amplification of cDNA end (RACE) primer ligated to all the 5′ capped mRNA ends. These cDNA libraries were used for subsequent experiments to generate specific TCR α and β chains.

TCR α and β chain variable region cDNAs were cloned by a 5′-RACE method (GeneRacer Kit, Invitrogen) as described (Zhao et al. (2006) J. IMMUNOTHR. 29: 398-406; Johnson et al. (2006) J. IMMUNOL. 177: 6548-6559; Morgan et al. (2006) SCIENCE 314: 126-129). Briefly, a 5′ RACE primer (5′-CGACTGGAGCACGAGGACACTGA-3′ (SEQ ID NO: 104)) was used together with a gene-specific 3′ primer (5′-GTTAACTAGTTCAGCTGGACCACAGCCGCAGC-3′(SEQ ID NO: 105), 5′-CGGGTTAACTAGTTCAGAAATCCTTTCTCTTGACCATGGC-3′(SEQ ID NO: 106), or 5′-CTAGCCTCTGGAATCCTTTCTCTTG-3′(SEQ ID NO: 107)) to enrich cDNA for TCR α, TCR β1 or TCR β2 chains, respectively. A second round of PCR followed using a 5′ nested PCR primer plus the original or a nested 3′ primer. The TCR α and β chain variable regions were both expected to be approximately 500 bp in length. The PCR products were then purified using a PCR purification kit (Qiagen, Germantown, MD) and subcloned into pCR2.1 TOPO vector (Invitrogen), followed by DNA sequencing to identify the relative frequencies of individual T-cell receptor α- and β-chain genes.

The T cell receptor amino acid sequences are as follows: SEQ ID NOS: 83 and 85 represent the amino acid sequences of the variable regions of the 806 T-cell receptor α-chain and β-chains, respectively, SEQ ID NOs: 7, 5, and 6 represent the amino acid sequences of the CDR₃, CDR₁ and CDR₂ sequences of the 806 T-cell receptor α-chain, and SEQ ID NOS: 13, 11, and 12 represent the amino acid sequences of the CDR₃, CDR₁ and CDR₂ sequences of the 806 T-cell receptor β-chain.

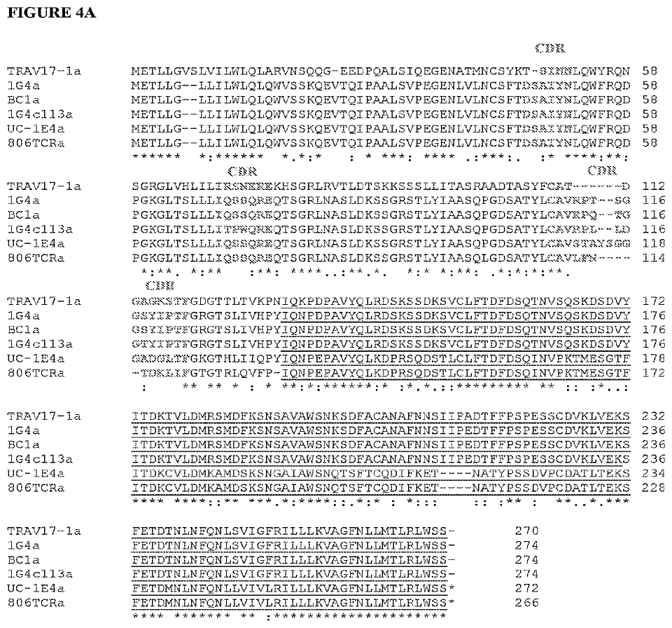

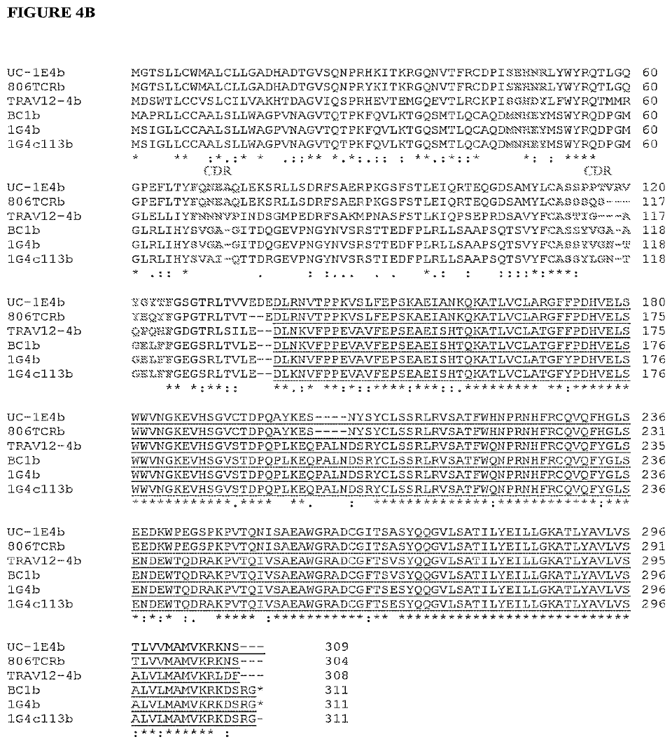

The amino acid and nucleotide sequences of the 806 TCR α/β chains were compared with sequences from two groups of known TCR α/β chains in publicly available databases, as shown in FIG. 4.

### Example 3—Mutagenesis of 806 T-Cell Receptor

As described above, mutagenesis of a TCR may increase the affinity, specificity, membrane targeting and expression levels of the TCR, which might be beneficial in clinical applications.

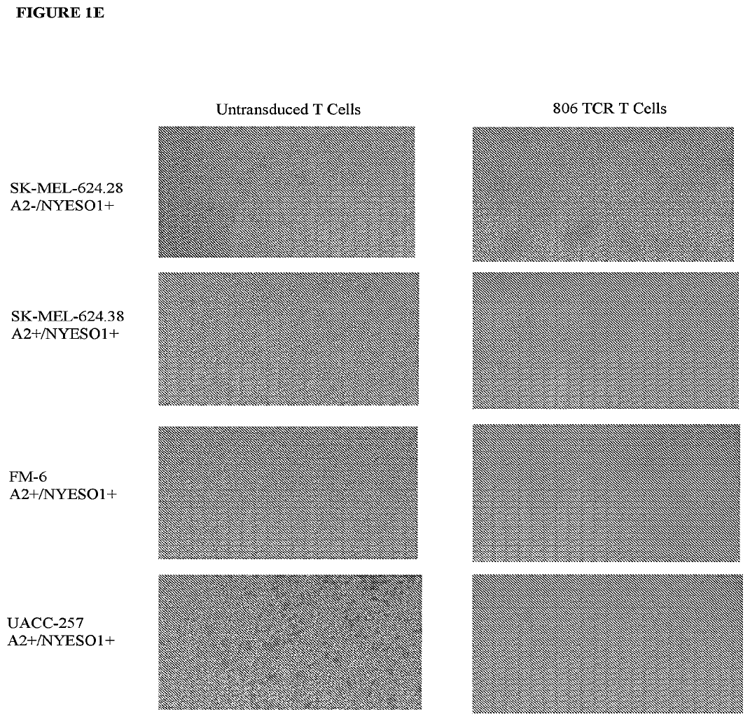

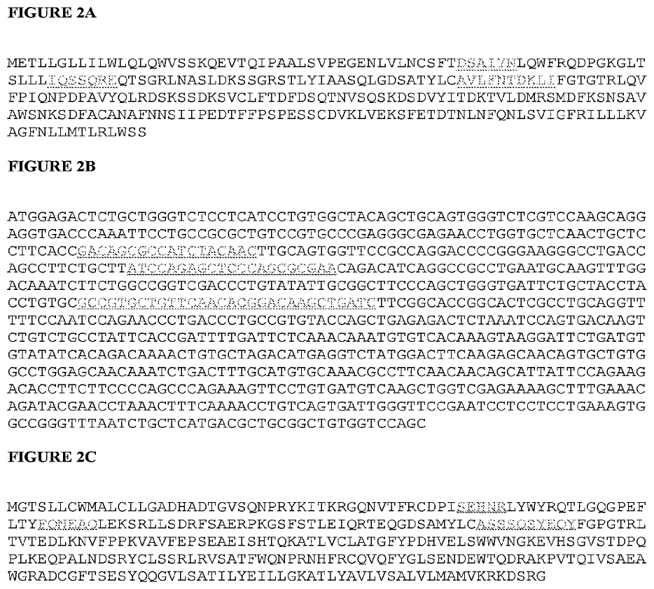

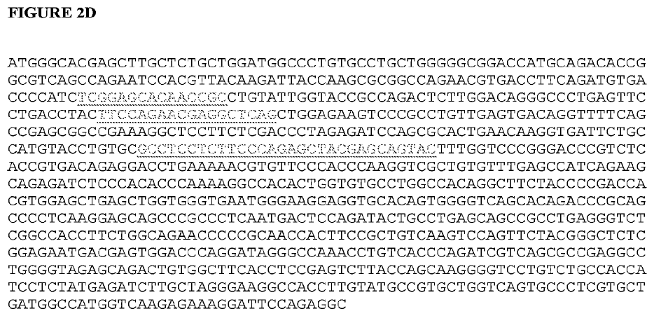

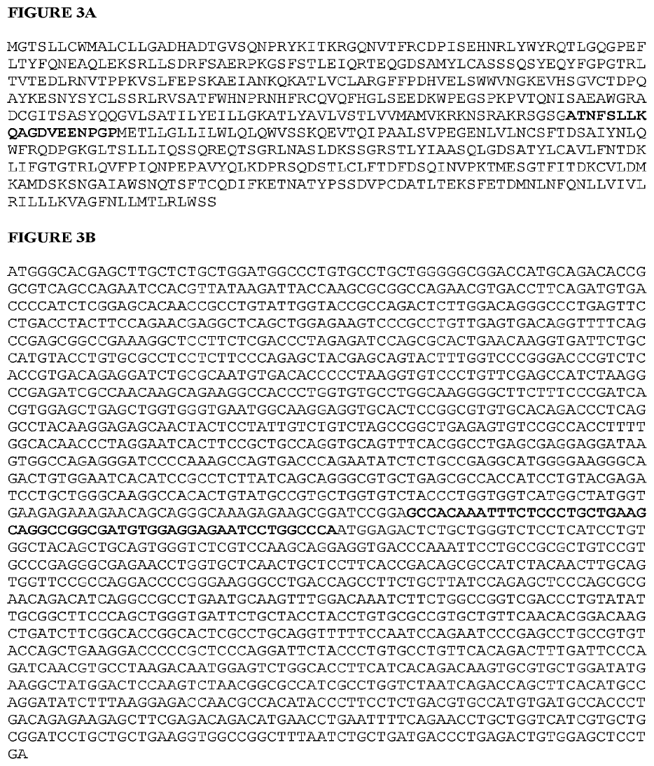

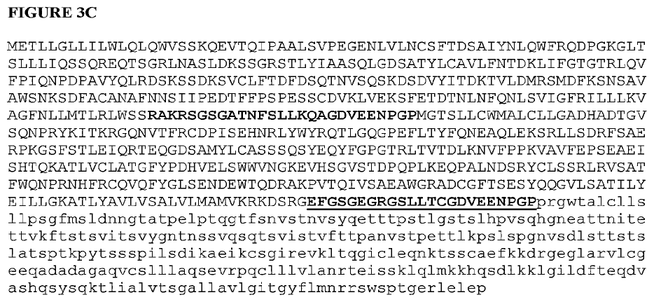

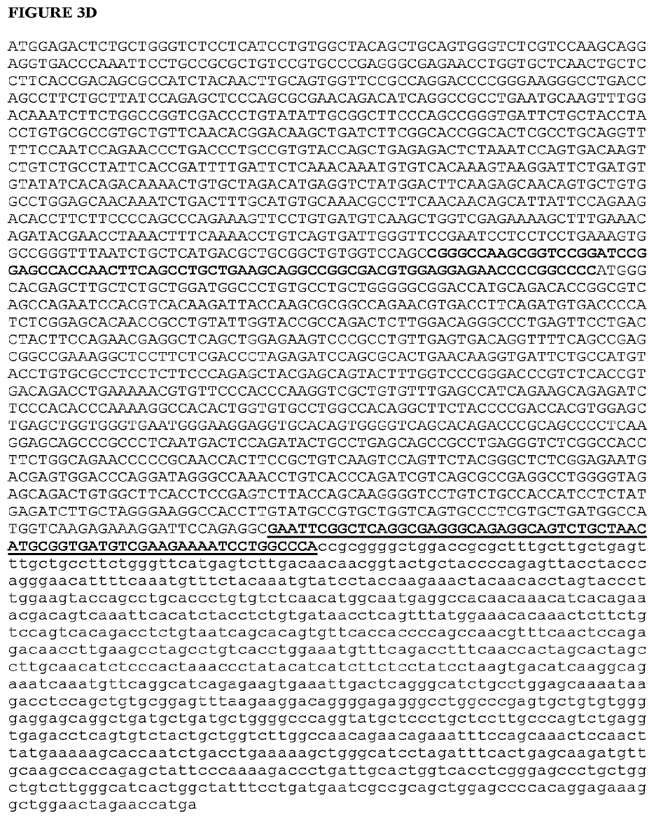

A particular engineered 806TCR included a P to L substitution at position 101 of the α-chain and an H to Y substitution at position 28 of the β-chain. The amino acid and nucleotide (codon optimized) sequences of this T-cell receptor are set forth in FIG. 2. SEQ ID NOS: 3 and 9 represent the amino acid sequences of the variable regions of the α-chain and β-chains, respectively, SEQ ID NOs: 7, 5, and 6 represent the amino acid sequences of the CDR₃, CDR₁ and CDR₂ sequences of the T-cell receptor α-chain, and SEQ ID NOS: 13, 11, and 12 represent the amino acid sequences of the CDR₃, CDR₁ and CDR₂ sequences of the T-cell receptor β-chain. SEQ ID NOs: 2 and 95 represent the amino acid sequences of a full-length receptor α-chain and β-chain, respectively, each including a human constant region. Certain foregoing amino acid sequences and the nucleotide sequences encoding such sequences are set forth in TABLE 1 above.

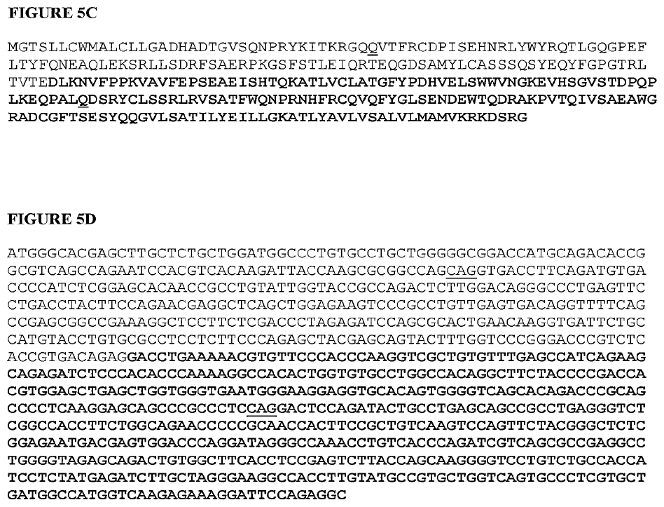

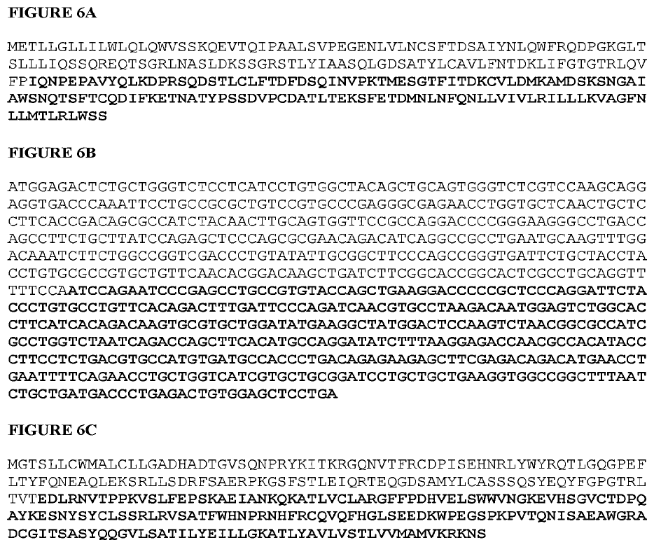

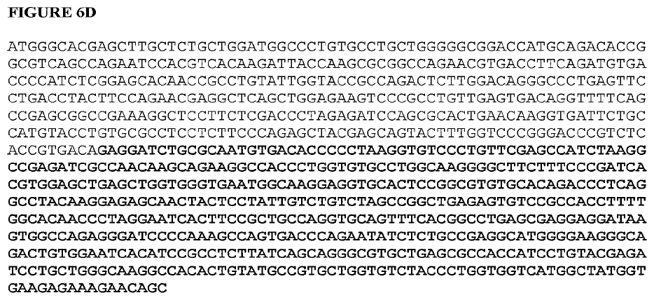

Additional potential mutations include, for example, a change in the key glycosylation site NXS/T to QXS/T (N→Q) in the constant region of the α- and/or β-chains. FIG. 5 depicts exemplary N→Q amino acid mutations in the 806 TCR α-chain and β-chain. An additional modification includes the use of murine constant regions, because pairing between murine TCR constant regions may reduce the mispairing of transduced T-cell receptors with endogenous T-cell receptors. FIG. 6 depicts amino acid and nucleotide sequences of 806 TCR α- and β-chains including an exemplary murine constant region. FIG. 7 depicts amino acid and nucleotide sequences of a 806 TCR α-chain that includes both a murine constant region and N→Q amino acid mutations.

### Example 4—Further Characterization Of 806 T-Cell Receptor

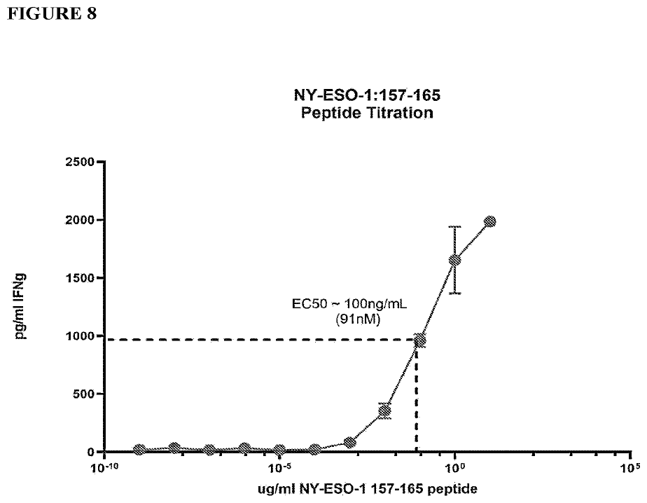

HLA-A*02:01+ antigen presenting cells were pulsed with NY-ESO-1:157-165 peptide in 10-fold dilutions starting at 10 μg/mL (9.1 μM). Donor T cells were transduced with a lentiviral expression vector encoding the 806TCR (including an α-chain variable region amino acid sequence of SEQ ID NO: 3, a β-chain variable region amino acid sequence of SEQ ID NO: 9, and a murine constant region). The pulsed APCs were co-cultured with the transduced T cells for 16 hours. Interferon-γ release was measured by ELISA. Results are depicted in FIG. 8. The EC50 was approximately 100 ng/mL (90.1 nM) for the 806TCR, with activity detectable at approximately 1 ng/mL (0.91 nM).

### Example 5—Cancer Cell Killing Mediated By The 806 T-Cell Receptor

This Example describes killing of target cancer cells by T cells expressing the 806 TCR (“806TCR-T cells”).

MEL-624.38 cells (NY-ESO-1+, HLA-A*02:01+) were transduced with a red fluorescent protein (RFP) nuclear marker. 806TCR-T cells were generated by transducing donor T cells from two donors (Donor 1 and Donor 2) with a lentiviral expression vector encoding the 806TCR (including an α-chain variable region amino acid sequence of SEQ ID NO: 3, a β-chain variable region amino acid sequence of SEQ ID NO: 9, and a murine constant region).

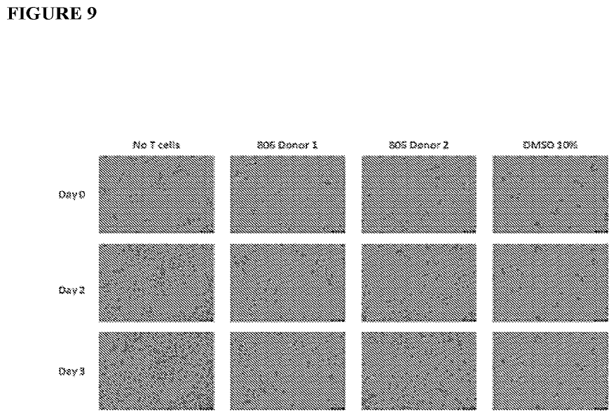

MEL-624.38 cells were plated at equal concentration. After 24 hours, 806TCR-T cells were added and co-cultured with the MEL-624.38 cells. All wells contained equal concentrations of cells at time 0. Cells were monitored by fluorescence microscopy, and images are depicted in FIG. 9. As depicted, 806TCR-T cells reduced the number of cancer cells relative to controls.

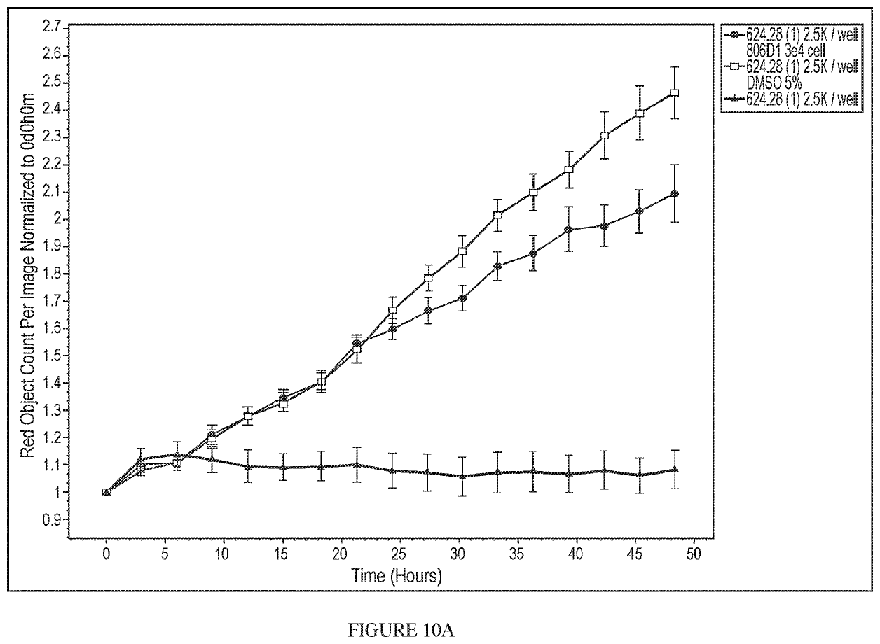

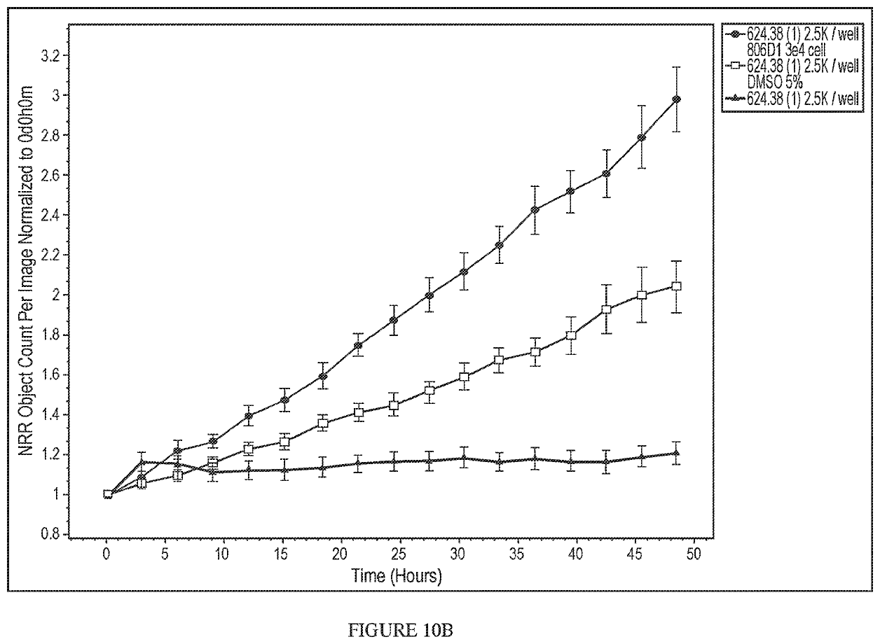

Further images were collected over 48 hours, and analyzed to count cell nuclei from an integrated area of target cells over time. Results are shown in FIG. 10. As depicted, co-culture of target cells (MEL-624.38 cells; NY-ESO-1+, HLA-A*02:01+) with 806TCR-T cells resulted in cancer cell killing, as indicated by a reduction in the number of cell nuclei over time relative to control. However, co-culture of off-target cells (MEL-624.28 cells; NY-ESO-1+ and HLA-A*02:01−) with 806TCR-T cells did not result in cancer cell killing.

Together, these results show that T cells expressing the 806 TCR (“806TCR-T cells”) can specifically kill target cancer cells.

### Example 6—Specificity Of 806 T-Cell Receptor Activity

This Example demonstrates a lack of off-target killing activity for T cells expressing the 806 TCR (“806TCR-T cells”).

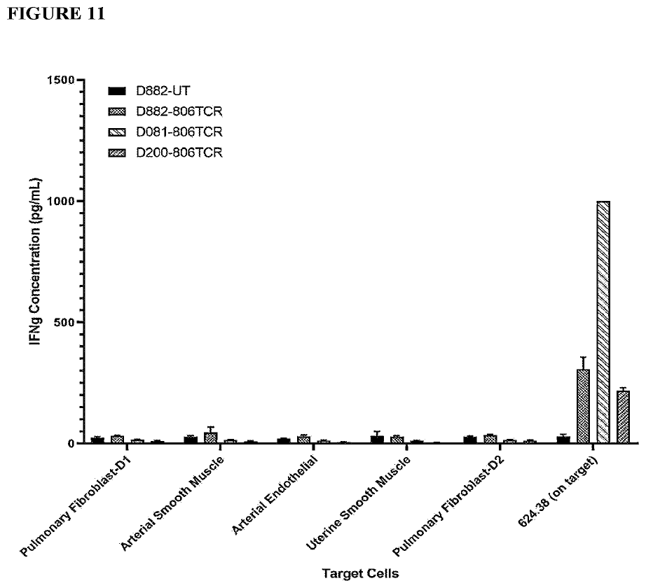

806TCR-T cells were generated by transducing donor T cells with a lentiviral expression vector encoding the 806TCR (including an α-chain variable region amino acid sequence of SEQ ID NO: 3, a β-chain variable region amino acid sequence of SEQ ID NO: 9, and a murine constant region). 806TCR-T cells were co-cultured with 4 non-cancerous cell types: pulmonary fibroblasts (2 donors), arterial smooth muscle, arterial endothelial cells, and uterine smooth muscle. Target cancer cells (MEL-624.38; NY-ESO-1+, HLA-A*02:01+) were also included as a positive control. Cells were co-cultured for 16 hours and interferon-gamma secretion was assayed by ELISA. Results are shown in FIG. 11. As depicted, no activity was observed following co-culture of 806TCR-expressing T cells with normal, non-cancerous cells. Activity was only observed following co-culture with the on-target cancer cells.

Together, these results show that T cells expressing the 806 TCR (“806TCR-T cells”) can specifically kill target cancer cells.

### Example 7-806 T-Cell Receptor Binding Affinity

This Example demonstrates measurement of the binding affinity of the 806TCR for a peptide/MHC complex by surface plasmon resonance.

A soluble 806 TCR (including an α-chain variable region amino acid sequence of SEQ ID NO: 83, a β-chain variable region amino acid sequence of SEQ ID NO: 85) was generated by expressing the 806 TCR α-chain and β-chain variable regions linked together (and without constant region) in mammalian cells. For comparison, a soluble 1G4LY TCR was also generated. 1G4LY TCR (described in Robbins et al. (2008) J. IMMUNOL. 180:6116-6131 and Robbins et al. (2011) J. CLIN. ONCOL. 29(7):917-924) is an affinity enhanced version of the 1G4 TCR (described in US. Patent Application Publication No. US2009/053184).

Ligand (pMHC1 complex: Biotin-HLA-A*02:01-SLLMWITQC (SEQ ID NO: 1)) was immobilized onto a streptavidin (SA) sensor chip surface with an immobilization level of about 400 RU. Then, the analytes (soluble TCRs) at concentrations of 250, 125, 62.5, 31.25, 15.625, 7.813, 3.906, 1.953, and 0 nM were injected onto the sensor surface.

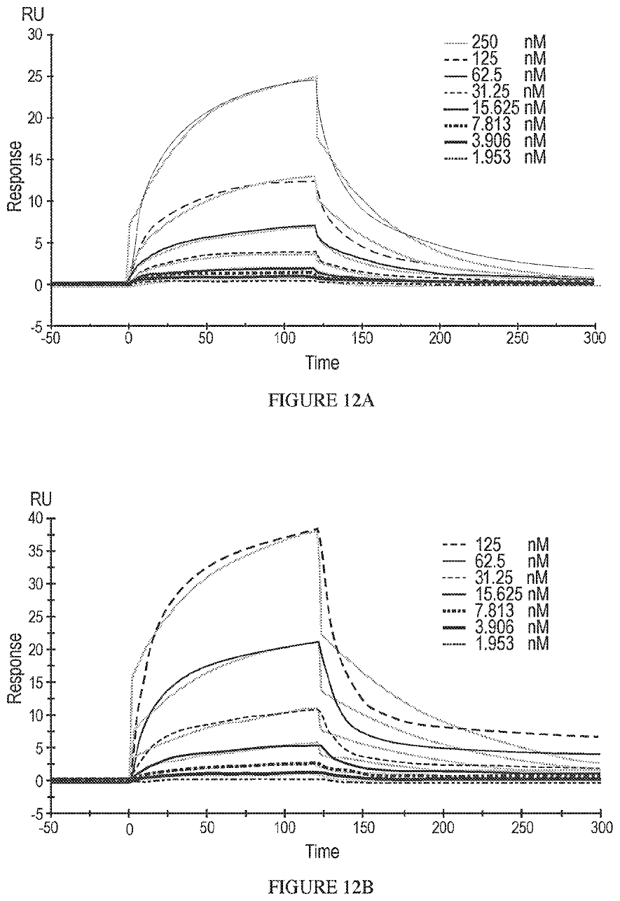

Results for 1G4LY TCR are depicted in FIG. 12A. A 1:1 binding model was used to measure the binding affinity and/or kinetics. For 1G4LY TCR the equilibrium dissociation constant (KD) was 7.61×10⁻⁷ M, the association rate constant (Ka) was 3.98×10⁴ M⁻¹s⁻¹, and the dissociation rate constant (Kd) was 3.03×10⁻²s⁻¹. These results are consistent with what has been previously reported (Robbins et al. (2008) J IMMUNOL 180:6116-6131). As expected, 1G4LY TCR had a higher binding affinity (lower KD) than wild-type 1G4 TCR (KD=32 μM).

Results for 806 TCR are depicted in FIG. 12B. A 1:1 binding model was used to measure the binding affinity and/or kinetics. For 806 TCR, the equilibrium dissociation constant (KD) was 1.34×10⁻⁷ M, the association rate constant (Ka) was 8.92×10⁴ M⁻¹s⁻¹, and the dissociation rate constant (Kd) was 1.20×10⁻² s⁻¹.

Together, these results show that 806 TCR has a higher binding affinity (lower KD) than the wild-type 1G4 TCR or the affinity-enhanced 1G4LY TCR, and that 806 TCR has a binding affinity that is in a range that is generally associated with high avidity (see, for example, Zhong et al. (2013) PNAS 110 (17):6973-6978, and Aleksic et al. (2012) EUR J IMMUNOL 42:3174-3179).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. An isolated recombinant α-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28), wherein the α-chain comprises one or more of the following amino acid sequences:
(i) an amino acid sequence of SEQ ID NO: 37, SEQ ID NO: 2, or SEQ ID NO: 87, or an amino acid sequence having greater than 97% identity to the amino acid sequence of SEQ ID NO: 37, SEQ ID NO: 2, or SEQ ID NO: 87;
(ii) an α-chain variable region amino acid sequence of SEQ ID NO: 3 or SEQ ID NO: 83, or an amino acid sequence having greater than 96% identity to the amino acid sequence of SEQ ID NO: 3 or SEQ ID NO: 83;
(iii) an α-chain CDR₃ amino acid sequence of SEQ ID NO: 7;
(iv) an α-chain CDR₁ amino acid sequence of SEQ ID NO: 5; and
(v) an α-chain CDR₂ amino acid sequence of SEQ ID NO: 6.

2. An isolated recombinant β-chain of a T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28), wherein the β-chain comprises one or more of the following amino acid sequences:
(i) an amino acid sequence of SEQ ID NO: 39, SEQ ID NO: 95, or SEQ ID NO: 99, or an amino acid sequence having greater than 97% identity to the amino acid sequence of SEQ ID NO: 39, SEQ ID NO: 95, or SEQ ID NO: 99;
(ii) a β-chain variable region amino acid sequence of SEQ ID NO: 9 or SEQ ID NO: 85, or an amino acid sequence having greater than 97% identity to the amino acid sequence of SEQ ID NO: 9 or SEQ ID NO: 85;
(iii) a β-chain CDR₃ amino acid sequence of SEQ ID NO: 13;
(iv) a β-chain CDR₁ amino acid sequence of SEQ ID NO: 11; and
(v) a β-chain CDR₂ amino acid sequence of SEQ ID NO: 12.

3. (canceled)

4. An isolated, recombinant T-cell receptor immunoreactive with an epitope of a NY-ESO-1 and/or LAGE-1a protein comprising the amino acid sequence SLLMWITQC (SEQ ID NO: 1) and/or SLLMWITQCFL (SEQ ID NO: 28), the T-cell receptor comprising an α-chain and a β-chain, the α and β chains each comprising a CDR₁, CDR₂, and a CDR₃, wherein the α-chain CDR₃ comprises the amino acid sequence of SEQ ID NO: 7, and the β-chain CDR₃ comprises the amino acid sequence of SEQ ID NO: 13.

5. The T-cell receptor of claim 4, wherein:
(i) the α-chain CDR₁ comprises the amino acid sequence of SEQ ID NO: 5, and the β-chain CDR₁ comprises the amino acid sequence of SEQ ID NO: 11;
(ii) the α-chain CDR₂ comprises the amino acid sequence of SEQ ID NO: 6, and the β-chain CDR₂ comprises the amino acid sequence of SEQ ID NO: 12;
(iii) the T-cell receptor comprises an α-chain variable region comprising an amino acid sequence of SEQ ID NO: 3 or SEQ ID NO: 83, or an amino acid sequence having greater than 96% identity to the amino acid sequence of SEQ ID NO: 3 or SEQ ID NO: 83;
(iv) the T-cell receptor comprises a β-chain variable region comprising an amino acid sequence of SEQ ID NO: 9 or SEQ ID NO: 85, or an amino acid sequence having greater than 97% identity to the amino acid sequence of SEQ ID NO: 9 or SEQ ID NO: 85;
(v) the α-chain comprises the amino acid sequence of SEQ ID NO: 37, SEQ ID NO: 2, or SEQ ID NO: 87, or an amino acid sequence having greater than 97% identity to the amino acid sequence of SEQ ID NO: 37, SEQ ID NO: 2, or SEQ ID NO: 87; and/or
(vi) the β-chain comprises the amino acid sequence of SEQ ID NO: 39, SEQ ID NO: 95, or SEQ ID NO: 99, or an amino acid sequence having greater than 97% identity to the amino acid sequence of SEO ID NO: 39, SEO ID NO: 95, or SEO ID NO: 99.

6-8. (canceled)

9. The T-cell receptor of claim 5, wherein the immunoreactivity to the epitope SLLMWITQC (SEQ ID NO: 1) and/or SLLMWITQCFL (SEQ ID NO: 28) is:
(a) HLA-A2 restricted; and/or
(b) HLA-A*0201 or HLA-A*0202 restricted.

10. (canceled)

11. The T-cell receptor of claim 9, wherein the T-cell receptor is a single chain T-cell receptor, optionally where the α-chain is linked to the β-chain via an amino acid linker.

12. The T-cell receptor of claim 11, further comprising a detectable label.

13. The T-cell receptor of claim 12, associated with a therapeutic agent.

14. A bispecific T-cell receptor protein comprising an antibody or an antigen binding fragment thereof associated with, optionally fused to, the T-cell receptor of claim 4.

15. (canceled)

16. An isolated recombinant nucleic acid encoding the T-cell receptor α-chain of claim 1.

17. An isolated recombinant nucleic acid encoding the T-cell receptor β-chain of claim 2.

18. An isolated recombinant nucleic acid encoding the T-cell receptor of claim 4.

19-22. (canceled)

23. The the T-cell receptor of claim 4, wherein the α-chain, the β-chain, or both the α- and β-chains comprise a mutation not present in the naturally occurring T-cell receptor, optionally including a point mutation to remove at least one glycosylation site in the α-chain, the β-chain, or both the α- and β-chains.

24. A recombinant expression vector comprising one or more of the nucleic acids of claim 18.

25-26. (canceled)

27. A genetically modified cell that comprises the T-cell receptor of claim 4.

28-33. (canceled)

34. A method for producing a T-cell immunoreactive with an epitope of an NY-ESO-1 and/or LAGE-1a protein and/or with an epitope of a LAGE-1a protein, the method comprising introducing one or more of the nucleic acids of claim 18 into the T-cell.

35-37. (canceled)

38. A pharmaceutical composition comprising the cell of claim 27.

39. A method of inhibiting the growth of cancer cells expressing an NY-ESO-1 and/or LAGE-1a protein, the method comprising exposing the cancer cells to a cell of claim 27 capable of inhibiting the growth of the cancer cells.

40. A method for treating or preventing cancer in a subject, the method comprising administering to the subject autologous genetically modified T-cells expressing the T-cell receptor of claim 4 in an amount effective to treat or prevent cancer in the subject.

41. A method for treating or preventing cancer in a subject, said method comprising the steps of
(i) extracting T-cells from the subject;
(ii) introducing into the T-cells one or more nucleic acids of claim 18; and
(iii) administering the T-cells produced by step (ii) to the subject.

42-43. (canceled)

44. The T-cell receptor of claim 4, wherein the α-chain comprises the amino acid sequence of SEQ ID NO: 37 and the β-chain comprises the amino acid sequence of SEQ ID NO: 39.

45. The T-cell receptor of claim 4, wherein the α-chain comprises the amino acid sequence of SEQ ID NO: 2 and the β-chain comprises the amino acid sequence of SEQ ID NO: 95.

46. The T-cell receptor of claim 4, wherein the α-chain comprises the amino acid sequence of SEQ ID NO: 87 and the β-chain comprises the amino acid sequence of SEQ ID NO: 99.


SEQUENCE LISTING (117 sequences)

SEQ ID NO: 1 - protein, 9 aa, Artificial Sequence
  Description of Artificial Sequence Synthetic peptide
  NY-ESO-1157-165
SLLMWITQC

SEQ ID NO: 2 - protein, 270 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQLGDSATYLCAVLFNTDKLIFGTGTRLQVFPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 130 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQLGDSATYLCAVLFNTDKLIFGTGTRLQVFP

SEQ ID NO: 4 - protein, 140 aa, Artificial Sequence
  Description of Artificial Sequence Synthetic polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 6 aa, Artificial Sequence
  Description of Artificial Sequence Synthetic peptide
  CDR1
DSAIYN

SEQ ID NO: 6 - protein, 7 aa, Artificial Sequence
  Description of Artificial Sequence Synthetic peptide
  CDR2
IQSSQRE

SEQ ID NO: 7 - protein, 10 aa, Artificial Sequence
  Description of Artificial Sequence Synthetic peptide
  CDR3
AVLFNTDKLI

SEQ ID NO: 8 - protein, 309 aa, Artificial Sequence
  Description of Artificial Sequence Synthetic polypeptide
MGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 9 - protein, 131 aa, Artificial Sequence
  Description of Artificial Sequence Synthetic polypeptide
MGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVT

SEQ ID NO: 10 - protein, 178 aa, Artificial Sequence
  Description of Artificial Sequence Synthetic polypeptide
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 5 aa, Artificial Sequence
  Description of Artificial Sequence Synthetic peptide
  CDR1
SEHNR

SEQ ID NO: 12 - protein, 6 aa, Artificial Sequence
  Description of Artificial Sequence Synthetic peptide
  CDR2
FQNEAQ

SEQ ID NO: 13 - protein, 10 aa, Artificial Sequence
  Description of Artificial Sequence Synthetic peptide
  CDR3
ASSSQSYEQY

SEQ ID NO: 14 - protein, 597 aa, Artificial Sequence
  Description of Artificial Sequence Synthetic polypeptide
MGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQLGDSATYLCAVLFNTDKLIFGTGTRLQVFPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 15 - DNA, 810 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttccaatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 16 - DNA, 390 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttcca

SEQ ID NO: 17 - DNA, 420 bp, Artificial Sequence
  Description of Artificial Sequence Synthetic polynucleotide
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 18 - DNA, 18 bp, Artificial Sequence
  Description of Artificial Sequence Synthetic oligonucleotide
gacagcgccatctacaac

SEQ ID NO: 19 - DNA, 21 bp, Artificial Sequence
  Description of Artificial Sequence Synthetic oligonucleotide
atccagagctcccagcgcgaa

SEQ ID NO: 20 - DNA, 30 bp, Artificial Sequence
  Description of Artificial Sequence Synthetic oligonucleotide
gccgtgctgttcaacacggacaagctgatc

SEQ ID NO: 21 - DNA, 927 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 22 - DNA, 393 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgaca

SEQ ID NO: 23 - DNA, 534 bp, Artificial Sequence
  Description of Artificial Sequence Synthetic polynucleotide
gacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 24 - DNA, 15 bp, Artificial Sequence
  Description of Artificial Sequence Synthetic oligonucleotide
tcggagcacaaccgc

SEQ ID NO: 25 - DNA, 18 bp, Artificial Sequence
  Description of Artificial Sequence Synthetic oligonucleotide
ttccagaacgaggctcag

SEQ ID NO: 26 - DNA, 30 bp, Artificial Sequence
  Description of Artificial Sequence Synthetic oligonucleotide
gcctcctcttcccagagctacgagcagtac

SEQ ID NO: 27 - DNA, 1794 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgttataagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggatctgcgcaatgtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagcaactactcctattgtctgtctagccggctgagagtgtccgccaccttttggcacaaccctaggaatcacttccgctgccaggtgcagtttcacggcctgagcgaggaggataagtggccagagggatccccaaagccagtgacccagaatatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagcagggcaaagagaagcggatccggagccacaaatttctccctgctgaagcaggccggcgatgtggaggagaatcctggcccaatggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagctgggtgattctgctacctacctgtgcgccgtgctgttcaacacggacaagctgatcttcggcaccggcactcgcctgcaggtttttccaatccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctaatcagaccagcttcacatgccaggatatctttaaggagaccaacgccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcctga

SEQ ID NO: 28 - protein, 11 aa, Artificial Sequence
  Description of Artificial Sequence Synthetic peptide
  NY-ESO-1157-167
SLLMWITQCFL

SEQ ID NO: 29 - protein, 944 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQLGDSATYLCAVLFNTDKLIFGTGTRLQVFPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGATNFSLLKQAGDVEENPGPMGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGEFGSGEGRGSLLTCGDVEENPGPPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 30 - DNA, 2835 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttccaatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagccgggccaagcggtccggatccggagccaccaacttcagcctgctgaagcaggccggcgacgtggaggagaaccccggccccatgggcacgagcttgctctgctggatggccctgtgcctgctgggggcggaccatgcagacaccggcgtcagccagaatccacgtcacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcgaattcggctcaggcgagggcagaggcagtctgctaacatgcggtgatgtcgaagaaaatcctggcccaccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgagacctcagtgtctactgctggtcttggccaacagaacagaaatttccagcaaactccaacttatgaaaaagcaccaatctgacctgaaaaagctgggcatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggaactagaaccatga

SEQ ID NO: 31 - protein, 270 aa, Artificial Sequence
  Description of Artificial Sequence Synthetic polypeptide
  806 alpha
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPNIQNPDPAVYQLRDSKSSDKSVCLFTDFDSQTNVSQSKDSDVYITDKTVLDMRSMDFKSNSAVAWSQKSDFACANAFNNSIIPEDTFFPSPESSCDVKLVEKSFETDTNLNFQNLSVIGFRILLLKVAGFNLLMTLRLWSS

SEQ ID NO: 32 - DNA, 813 bp, Artificial Sequence
  Description of Artificial Sequence Synthetic polynucleotide
  806 alpha
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttccgaacattcagaacccggatccggcggtgtatcagctgcgcgatagcaaaagcagcgataaaagcgtgtgcctgtttaccgattttgatagccagaccaacgtgagccagagcaaagatagcgatgtgtatattaccgataaaaccgtgcaggatatgcgcagcatggattttaaaagcaacagcgcggtggcgtggagccaaaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagctaa

SEQ ID NO: 33 - protein, 270 aa, Artificial Sequence
  Description of Artificial Sequence Synthetic polypeptide
  806 alpha
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPNIQNPDPAVYQLRDSKSSDKSVCLFTDFDSQTQVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - DNA, 813 bp, Artificial Sequence
  Description of Artificial Sequence Synthetic polynucleotide
  806 alpha
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttccgaacattcagaacccggatccggcggtgtatcagctgcgcgatagcaaaagcagcgataaaagcgtgtgcctgtttaccgattttgatagccagacccaagtgagccagagcaaagatagcgatgtgtatattaccgataaaaccgtgcaggatatgcgcagcatggattttaaaagcaacagcgcggtggcgtggagcaacaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagctaa

SEQ ID NO: 35 - protein, 309 aa, Artificial Sequence
  Description of Artificial Sequence Synthetic polypeptide
  806 beta
MGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLNKVFPPEVAVFEPSEAEISHTQKATLVCLATGFFPDHVELSWWVNGKEVHSGVSTDPQPLKEQPALQDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRG

SEQ ID NO: 36 - DNA, 930 bp, Artificial Sequence
  Description of Artificial Sequence Synthetic polynucleotide
  806 beta
atgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgaccgaagatctgaaaaacgtgtttccgccggaagtggcgatgtttgaaccgagcgaagcggaaattagccatacccagaaagcgaccctggtgtgcctggcgaccggcttttatccggatcatgtggaactgagctggtgggtgaacggcaaagaagtgcatagcggcgtgagcaccgatccgcagccgctgaaagaacagccggcgctgcaagatagccgctattgcctgagcagccgcctgcgcgtgagcgcgaccttttggcagaacccgcgcaaccattttcgctgccaggtgcagttttatggcctgagcgaaaacgatgaatggacccaggatcgcgcgaaaccggtgacccagattgtgagcgcggaagcgtggggccgcgcggattgcggctttaccagcgaaagctatcagcagggcgtgctgagcgcgaccattctgtatgaaattctgctgggcaaagcgaccctgtatgcggtgctggtgagcgcgctggtgctgatggcgatggtgaaacgcaaagatagccgcggctaa

SEQ ID NO: 37 - protein, 266 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQLGDSATYLCAVLFNTDKLIFGTGTRLQVFPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 38 - DNA, 801 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttccaatccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctaatcagaccagcttcacatgccaggatatctttaaggagaccaacgccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcctga

SEQ ID NO: 39 - protein, 304 aa, Artificial Sequence
  Description of Artificial Sequence Synthetic polypeptide
MGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 40 - DNA, 912 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggatctgcgcaatgtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagcaactactcctattgtctgtctagccggctgagagtgtccgccaccttttggcacaaccctaggaatcacttccgctgccaggtgcagtttcacggcctgagcgaggaggataagtggccagagggatccccaaagccagtgacccagaatatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagc

SEQ ID NO: 41 - protein, 266 aa, Artificial Sequence
  Description of Artificial Sequence Synthetic polypeptide
  806 alpha
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPDIQNPEPAVYQLKDPRSQDSTLCLFTDFDSQINVPKTMESGTFITDKTVLDMKAMDSKSNGAIAWSQQTSFTCQDIFKETNATYPSSDVPCDATLTEKSFETDMNLNFQNLSVMGLRILLLKVAGFNLLMTLRLWSS

SEQ ID NO: 42 - DNA, 801 bp, Artificial Sequence
  Description of Artificial Sequence Synthetic polynucleotide
  806 alpha
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttcccgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagccaacagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagttga

SEQ ID NO: 43 - protein, 6 aa, Artificial Sequence
  Description of Artificial Sequence Synthetic peptide
  CDR1
DSAIYN

SEQ ID NO: 44 - protein, 5 aa, Artificial Sequence
  Description of Artificial Sequence Synthetic peptide
  CDR2
IQSSQ

SEQ ID NO: 45 - protein, 12 aa, Artificial Sequence
  Description of Artificial Sequence Synthetic peptide
  CDR3
CAVLFNTDKLIF

SEQ ID NO: 46 - protein, 5 aa, Artificial Sequence
  Description of Artificial Sequence Synthetic peptide
  CDR1
SEHNR

SEQ ID NO: 47 - protein, 6 aa, Artificial Sequence
  Description of Artificial Sequence Synthetic peptide
  CDR2
QNEAQL

SEQ ID NO: 48 - protein, 12 aa, Artificial Sequence
  Description of Artificial Sequence Synthetic peptide
  CDR3
CASSSQSYEQYF

SEQ ID NO: 49 - protein, 136 aa, Artificial Sequence
  Description of Artificial Sequence Synthetic polypeptide
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 50 - DNA, 411 bp, Artificial Sequence
  Description of Artificial Sequence Synthetic polynucleotide
atccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctaatcagaccagcttcacatgccaggatatctttaaggagaccaacgccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcctga

SEQ ID NO: 51 - protein, 173 aa, Artificial Sequence
  Description of Artificial Sequence Synthetic polypeptide
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 52 - DNA, 519 bp, Artificial Sequence
  Description of Artificial Sequence Synthetic polynucleotide
gaggatctgcgcaatgtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagcaactactcctattgtctgtctagccggctgagagtgtccgccaccttttggcacaaccctaggaatcacttccgctgccaggtgcagtttcacggcctgagcgaggaggataagtggccagagggatccccaaagccagtgacccagaatatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagc

SEQ ID NO: 53 - protein, 270 aa, Artificial Sequence
  Description of Artificial Sequence Synthetic polypeptide
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 128 aa, Artificial Sequence
  Description of Artificial Sequence Synthetic polypeptide
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVF

SEQ ID NO: 55 - protein, 142 aa, Artificial Sequence
  Description of Artificial Sequence Synthetic polypeptide
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 309 aa, Artificial Sequence
  Description of Artificial Sequence Synthetic polypeptide
MGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRG

SEQ ID NO: 57 - protein, 130 aa, Artificial Sequence
  Description of Artificial Sequence Synthetic polypeptide
MGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVT

SEQ ID NO: 58 - protein, 179 aa, Artificial Sequence
  Description of Artificial Sequence Synthetic polypeptide
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRG

SEQ ID NO: 59 - DNA, 813 bp, Artificial Sequence
  Description of Artificial Sequence Synthetic polynucleotide
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttccgaacattcagaacccggatccggcggtgtatcagctgcgcgatagcaaaagcagcgataaaagcgtgtgcctgtttaccgattttgatagccagaccaacgtgagccagagcaaagatagcgatgtgtatattaccgataaaaccgtgcaggatatgcgcagcatggattttaaaagcaacagcgcggtggcgtggagcaacaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagctaa

SEQ ID NO: 60 - DNA, 384 bp, Artificial Sequence
  Description of Artificial Sequence Synthetic polynucleotide
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgttt

SEQ ID NO: 61 - DNA, 428 bp, Artificial Sequence
  Description of Artificial Sequence Synthetic polynucleotide
ccgaacattcagaacccggatccggcggtgtatcagctgcgcgatagcaaaagcagcgataaaagcgtgtgcctgtttaccgattttgatagccagaccaacgtgagccagagcaaagatagcgatgtgtatattaccgataaaaccgtgcaggatatgcgcagcatggattttaaaagcaacagcgcggtggcgtggagcaacaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagcta

SEQ ID NO: 62 - DNA, 18 bp, Artificial Sequence
  Description of Artificial Sequence Synthetic oligonucleotide
gatagcgcgatttataac

SEQ ID NO: 63 - DNA, 15 bp, Artificial Sequence
  Description of Artificial Sequence Synthetic oligonucleotide
  806 alpha cdr2
attcagagcagccag

SEQ ID NO: 64 - DNA, 36 bp, Artificial Sequence
  Description of Artificial Sequence Synthetic oligonucleotide
tgcgcggtgctgtttaacaccgataaactgattttt

SEQ ID NO: 65 - DNA, 930 bp, Artificial Sequence
  Description of Artificial Sequence Synthetic polynucleotide
atgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgaccgaagatctgaaaaacgtgtttccgccggaagtggcgatgtttgaaccgagcgaagcggaaattagccatacccagaaagcgaccctggtgtgcctggcgaccggcttttatccggatcatgtggaactgagctggtgggtgaacggcaaagaagtgcatagcggcgtgagcaccgatccgcagccgctgaaagaacagccggcgctgaacgatagccgctattgcctgagcagccgcctgcgcgtgagcgcgaccttttggcagaacccgcgcaaccattttcgctgccaggtgcagttttatggcctgagcgaaaacgatgaatggacccaggatcgcgcgaaaccggtgacccagattgtgagcgcggaagcgtggggccgcgcggattgcggctttaccagcgaaagctatcagcagggcgtgctgagcgcgaccattctgtatgaaattctgctgggcaaagcgaccctgtatgcggtgctggtgagcgcgctggtgctgatggcgatggtgaaacgcaaagatagccgcggctaa

SEQ ID NO: 66 - DNA, 390 bp, Artificial Sequence
  Description of Artificial Sequence Synthetic polynucleotide
atgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgacc

SEQ ID NO: 67 - DNA, 540 bp, Artificial Sequence
  Description of Artificial Sequence Synthetic polynucleotide
gaagatctgaaaaacgtgtttccgccggaagtggcgatgtttgaaccgagcgaagcggaaattagccatacccagaaagcgaccctggtgtgcctggcgaccggcttttatccggatcatgtggaactgagctggtgggtgaacggcaaagaagtgcatagcggcgtgagcaccgatccgcagccgctgaaagaacagccggcgctgaacgatagccgctattgcctgagcagccgcctgcgcgtgagcgcgaccttttggcagaacccgcgcaaccattttcgctgccaggtgcagttttatggcctgagcgaaaacgatgaatggacccaggatcgcgcgaaaccggtgacccagattgtgagcgcggaagcgtggggccgcgcggattgcggctttaccagcgaaagctatcagcagggcgtgctgagcgcgaccattctgtatgaaattctgctgggcaaagcgaccctgtatgcggtgctggtgagcgcgctggtgctgatggcgatggtgaaacgcaaagatagccgcggctaa

SEQ ID NO: 68 - DNA, 15 bp, Artificial Sequence
  Description of Artificial Sequence Synthetic oligonucleotide
agcgaacataaccgc

SEQ ID NO: 69 - DNA, 18 bp, Artificial Sequence
  Description of Artificial Sequence Synthetic oligonucleotide
cagaacgaagcgcagctg

SEQ ID NO: 70 - DNA, 36 bp, Artificial Sequence
  Description of Artificial Sequence Synthetic oligonucleotide
tgcgcgagcagcagccagagctatgaacagtatttt

SEQ ID NO: 71 - protein, 266 aa, Artificial Sequence
  Description of Artificial Sequence Synthetic polypeptide
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 72 - DNA, 801 bp, Artificial Sequence
  Description of Artificial Sequence Synthetic polynucleotide
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttcccgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagttga

SEQ ID NO: 73 - protein, 302 aa, Artificial Sequence
  Description of Artificial Sequence Synthetic polypeptide
MGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRNVTPPKVSLFEPSKAEIANKQKATLVCLARGFFPDHVELSWWVNGKEVHSGVSTDPQAYKESNYSHCLSSRLRVSATFWHNPRNHFRCQVQFHGLSEEDKWPEGSPKPVTQNISAEAWGRADCGITSASYQQGVLSATILYEIQLGKATLYAVVSTLVVMAMVKRKNS

SEQ ID NO: 74 - DNA, 912 bp, Artificial Sequence
  Description of Artificial Sequence Synthetic polynucleotide
atgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgaccgaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagccactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatccagctagggaaagccaccctgtatgctgtgcttgtcagtacactggtggtgatggctatggtcaaaagaaagaattcctga

SEQ ID NO: 75 - protein, 270 aa, Artificial Sequence
  Description of Artificial Sequence Synthetic polypeptide
METLLGLLILWLQLQWVSSKQEVTQIPAALSVPEGENLVLQCSFTDSAIYNLQWFRQDPGKGLTSLLLIQSSQREQTSGRLQASLDKSSGRSTLYIAASQLGDSATYLCAVLFNTDKLIFGTGTRLQVFPIQNPDPAVYQLRDSKSSDKSVCLFTDFDSQTQVSQSKDSDVYITDKTVLDMRSMDFKSNSAVAWSQKSDFACANAFQNSIIPEDTFFPSPESSCDVKLVEKSFETDTNLNFQQLSVIGFRILLLKVAGFNLLMTLRLWSS

SEQ ID NO: 76 - DNA, 810 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctccagtgctccttcaccgacagcgccatctacaacttgcagtggttccgccaggaccccgggaagggcctgaccagccttctgcttatccagagctcccagcgcgaacagacatcaggccgcctgcaggcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttccaatccagaaccctgaccctgccgtgtaccagctgagagactctaaatccagtgacaagtctgtctgcctattcaccgattttgattctcaaacacaggtgtcacaaagtaaggattctgatgtgtatatcacagacaaaactgtgctagacatgaggtctatggacttcaagagcaacagtgctgtggcctggagccagaaatctgactttgcatgtgcaaacgccttccagaacagcattattccagaagacaccttcttccccagcccagaaagttcctgtgatgtcaagctggtcgagaaaagctttgaaacagatacgaacctaaactttcaacagctgtcagtgattgggttccgaatcctcctcctgaaagtggccgggtttaatctgctcatgacgctgcggctgtggtccagc

SEQ ID NO: 77 - protein, 309 aa, Artificial Sequence
  Description of Artificial Sequence Synthetic polypeptide
MGTSLLCWMALCLLGADHADTGVSQNPRYKITKRGQQVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTDLKNVFPPKVAVFEPSEAEISHTQKATLVCLATGFYPDHVELSWWVNGKEVHSGVSTDPQPLKEQPALQDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 78 - DNA, 927 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagcaggtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctccaggactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 79 - protein, 266 aa, Artificial Sequence
  Description of Artificial Sequence Synthetic polypeptide
METLLGLLILWLQLQWVSSKQEVTQIPAALSVPEGENLVLQCSFTDSAIYNLQWFRQDPGKGLTSLLLIQSSQREQTSGRLQASLDKSSGRSTLYIAASQLGDSATYLCAVLFNTDKLIFGTGTRLQVFPIQNPEPAVYQLKDPRSQDSTLCLFTDFDSQINVPKTMESGTFITDKCVLDMKAMDSKSNGAIAWSQQTSFTCQDIFKETQATYPSSDVPCDATLTEKSFETDMNLNFQNLLVIVLRILLLKVAGFNLLMTLRLWSS

SEQ ID NO: 80 - DNA, 801 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctccagtgctccttcaccgacagcgccatctacaacttgcagtggttccgccaggaccccgggaagggcctgaccagccttctgcttatccagagctcccagcgcgaacagacatcaggccgcctgcaggcaagtttggacaaatcttctggccggtcgaccctgtatattgcggcttcccagccgggtgattctgctacctacctgtgcgccgtgctgttcaacacggacaagctgatcttcggcaccggcactcgcctgcaggtttttccaatccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctcagcagaccagcttcacatgccaggatatctttaaggagacccaggccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcctga

SEQ ID NO: 81 - protein, 304 aa, Artificial Sequence
  Description of Artificial Sequence Synthetic polypeptide
MGTSLLCWMALCLLGADHADTGVSQNPRYKITKRGQQVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRQVTPPKVSLFEPSKAEIANKQKATLVCLARGFFPDHVELSWWVNGKEVHSGVCTDPQAYKESQYSYCLSSRLRVSATFWHNPRNHFRCQVQFHGLSEEDKWPEGSPKPVTQQISAEAWGRADCGITSASYQQGVLSATILYEILLGKATLYAVLVSTLVVMAMVKRKNS

SEQ ID NO: 82 - DNA, 912 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagcaggtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggatctgcgccaggtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagccagtactcctattgtctgtctagccggctgagagtgtccgccaccttttggcacaaccctaggaatcacttccgctgccaggtgcagtttcacggcctgagcgaggaggataagtggccagagggatccccaaagccagtgacccagcagatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagc

SEQ ID NO: 83 - protein, 130 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFP

SEQ ID NO: 84 - protein, 266 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 85 - protein, 131 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVT

SEQ ID NO: 86 - protein, 304 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 87 - protein, 270 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 309 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 89 - protein, 606 aa, Artificial Sequence
  Description of Artificial Sequence Synthetic polypeptide
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPNIQNPDPAVYQLRDSKSSDKSVCLFTDFDSQTNVSQSKDSDVYITDKTVQDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGATNFSLLKQAGDVEENPGPMGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLKNVFPPEVAMFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 90 - DNA, 1821 bp, Artificial Sequence
  Description of Artificial Sequence Synthetic polynucleotide
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttccgaacattcagaacccggatccggcggtgtatcagctgcgcgatagcaaaagcagcgataaaagcgtgtgcctgtttaccgattttgatagccagaccaacgtgagccagagcaaagatagcgatgtgtatattaccgataaaaccgtgcaggatatgcgcagcatggattttaaaagcaacagcgcggtggcgtggagcaacaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagccgggccaagcggtccggatccggagccaccaacttcagcctgctgaagcaggccggcgacgtggaggagaaccccggccccatgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgaccgaagatctgaaaaacgtgtttccgccggaagtggcgatgtttgaaccgagcgaagcggaaattagccatacccagaaagcgaccctggtgtgcctggcgaccggcttttatccggatcatgtggaactgagctggtgggtgaacggcaaagaagtgcatagcggcgtgagcaccgatccgcagccgctgaaagaacagccggcgctgaacgatagccgctattgcctgagcagccgcctgcgcgtgagcgcgaccttttggcagaacccgcgcaaccattttcgctgccaggtgcagttttatggcctgagcgaaaacgatgaatggacccaggatcgcgcgaaaccggtgacccagattgtgagcgcggaagcgtggggccgcgcggattgcggctttaccagcgaaagctatcagcagggcgtgctgagcgcgaccattctgtatgaaattctgctgggcaaagcgaccctgtatgcggtgctggtgagcgcgctggtgctgatggcgatggtgaaacgcaaagatagccgcggctaa

SEQ ID NO: 91 - protein, 944 aa, Artificial Sequence
  Description of Artificial Sequence Synthetic polypeptide
METLLGLLILWLQLQVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPNIQNPDPAVYQLRDSKSSDKSVCLFTDFDSQTNVSQSKDSDVYITDKTVQDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGATNFSLLKQAGDVEENPGPMGTSLLCWMALCLLGAHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLKNVFPPEVAMFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGEFGSGEGRGSLLTCGDVEENPGPPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 92 - DNA, 2835 bp, Artificial Sequence
  Description of Artificial Sequence Synthetic polynucleotide
atggaaaccctgctgggcctgctgattctgtggctgcagctgcaggtgagcagcaaacaggaagtgacccagattccggcggcgctgagcgtgccggaaggcgaaaacctggtgctgaactgcagctttaccgatagcgcgatttataacctgcagtggtttcgccaggatccgggcaaaggcctgaccagcctgctgctgattcagagcagccagcgcgaacagaccagcggccgcctgaacgcgagcctggataaaagcagcggccgcagcaccctgtatattgcggcgagccagccgggcgatagcgcgacctatctgtgcgcggtgctgtttaacaccgataaactgatttttggcaccggcacccgcctgcaggtgtttccgaacattcagaacccggatccggcggtgtatcagctgcgcgatagcaaaagcagcgataaaagcgtgtgcctgtttaccgattttgatagccagaccaacgtgagccagagcaaagatagcgatgtgtatattaccgataaaaccgtgcaggatatgcgcagcatggattttaaaagcaacagcgcggtggcgtggagcaacaaaagcgattttgcgtgcgcgaacgcgtttaacaacagcattattccggaagataccttttttccgagcccggaaagcagctgcgatgtgaaactggtggaaaaaagctttgaaaccgataccaacctgaactttcagaacctgagcgtgattggctttcgcattctgctgctgaaagtggcgggctttaacctgctgatgaccctgcgcctgtggagcagccgggccaagcggtccggatccggagccaccaacttcagcctgctgaagcaggccggcgacgtggaggagaaccccggccccatgggcaccagcctgctgtgctggatggcgctgtgcctgctgggcgcgcatgcggataccggcgtgagccagaacccgcgccataaaattaccaaacgcggccagaacgtgacctttcgctgcgatccgattagcgaacataaccgcctgtattggtatcgccagaccctgggccagggcccggaatttctgacctattttcagaacgaagcgcagctggaaaaaagccgcctgctgagcgatcgctttagcgcggaacgcccgaaaggcagctttagcaccctggaaattcagcgcaccgaacagggcgatagcgcgatgtatctgtgcgcgagcagcagccagagctatgaacagtattttggcccgggcacccgcctgaccgtgaccgaagatctgaaaaacgtgtttccgccggaagtggcgatgtttgaaccgagcgaagcggaaattagccatacccagaaagcgaccctggtgtgcctggcgaccggcttttatccggatcatgtggaactgagctggtgggtgaacggcaaagaagtgcatagcggcgtgagcaccgatccgcagccgctgaaagaacagccggcgctgaacgatagccgctattgcctgagcagccgcctgcgcgtgagcgcgaccttttggcagaacccgcgcaaccattttcgctgccaggtgcagttttatggcctgagcgaaaacgatgaatggacccaggatcgcgcgaaaccggtgacccagattgtgagcgcggaagcgtggggccgcgcggattgcggctttaccagcgaaagctatcagcagggcgtgctgagcgcgaccattctgtatgaaattctgctgggcaaagcgaccctgtatgcggtgctggtgagcgcgctggtgctgatggcgatggtgaaacgcaaagatagccgcggcgaattcggctcaggcgagggcagaggcagtctgctaacatgcggtgatgtcgaagaaaatcctggcccaccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgagacctcagtgtctactgctggtcttggccaacagaacagaaatttccagcaaactccaacttatgaaaaagcaccaatctgacctgaaaaagctgggcatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggaactagaaccatga

SEQ ID NO: 93 - protein, 944 aa, Artificial Sequence; identical to SEQ ID NO: 91
  Description of Artificial Sequence Synthetic polypeptide

SEQ ID NO: 94 - DNA, 2835 bp, Artificial Sequence; identical to SEQ ID NO: 92
  Description of Artificial Sequence Synthetic polynucleotide

SEQ ID NO: 95 - protein, 310 aa, Artificial Sequence
  Description of Artificial Sequence Synthetic polypeptide
MGTSLLCWMALCLLGADHADTGVSQNPRYKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 96 - DNA, 930 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 97 - protein, 310 aa, Artificial Sequence
  Description of Artificial Sequence Synthetic polypeptide
MGTSLLCWMALCLLGADHADTGVSQNPRYKITKRGQQVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLKNVFPPKVAVFEPSEAEISHTQKATLVCLATGFYPDHVELSWWVNGKEVHSGVSTDPQPLKEQPALQDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 98 - DNA, 930 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgtcacaagattaccaagcgcggccagcaggtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctccaggactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 99 - protein, 310 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 100 - DNA, 810 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagctgggtgattctgctacctacctgtgcgccgtgctgttcaacacggacaagctgatcttcggcaccggcactcgcctgcaggtttttccaatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 101 - DNA, 390 bp, Artificial Sequence
  Description of Artificial Sequence Synthetic polynucleotide
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccgacagcgccatctacaacttgcagtggttccgccaggaccccgggaagggcctgaccagccttctgcttatccagagctcccagcgcgaacagacatcaggccgcctgaatgcaagtttggacaaatcttctggccggtcgaccctgtatattgcggcttcccagctgggtgattctgctacctacctgtgcgccgtgctgttcaacacggacaagctgatcttcggcaccggcactcgcctgcaggtttttcca

SEQ ID NO: 102 - DNA, 930 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgttacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgacagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 103 - DNA, 393 bp, Artificial Sequence
  Description of Artificial Sequence Synthetic polynucleotide
atgggcacgagcttgctctgctggatggccctgtgcctgctgggggcggaccatgcagacaccggcgtcagccagaatccacgttacaagattaccaagcgcggccagaacgtgaccttcagatgtgaccccatctcggagcacaaccgcctgtattggtaccgccagactcttggacagggccctgagttcctgacctacttccagaacgaggctcagctggagaagtcccgcctgttgagtgacaggttttcagccgagcggccgaaaggctccttctcgaccctagagatccagcgcactgaacaaggtgattctgccatgtacctgtgcgcctcctcttcccagagctacgagcagtactttggtcccgggacccgtctcaccgtgaca

SEQ ID NO: 104 - DNA, 23 bp, Artificial Sequence
  Description of Artificial Sequence Synthetic primer
cgactggagcacgaggacactga

SEQ ID NO: 105 - DNA, 32 bp, Artificial Sequence
  Description of Artificial Sequence Synthetic primer
gttaactagttcagctggaccacagccgcagc

SEQ ID NO: 106 - DNA, 40 bp, Artificial Sequence
  Description of Artificial Sequence Synthetic primer
cgggttaactagttcagaaatcctttctcttgaccatggc

SEQ ID NO: 107 - DNA, 25 bp, Artificial Sequence
  Description of Artificial Sequence Synthetic primer
ctagcctctggaatcctttctcttg

SEQ ID NO: 108 - protein, 270 aa, Artificial Sequence
  Description of Artificial Sequence Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDGAGKSTFGDGTTLTVKPNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 109 - protein, 274 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 274 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPQTGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 111 - protein, 274 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 112 - protein, 272 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVSTAYSGGGADGLTFGKGTHLIIQPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 113 - protein, 309 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPPTVRVYGYTFGSGTRLTVVEDEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 114 - protein, 308 aa, Artificial Sequence
  Description of Artificial Sequence Synthetic polypeptide
MDSWTLCCVSLCILVAKHTDAGVIQSPRHEVTEMGQEVTLRCKPISGHDYLFWYRQTMMRGLELLIYFNNNVPINDSGMPEDRFSAKMPNASFSTLKIQPSEPRDSAVYFCASTIGAQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRLDF

SEQ ID NO: 115 - protein, 311 aa, Artificial Sequence
  Description of Artificial Sequence Synthetic polypeptide
MAPRLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGAAGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRG

SEQ ID NO: 116 - protein, 311 aa, Artificial Sequence
  Description of Artificial Sequence Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 117 - protein, 311 aa, Artificial Sequence
  Description of Artificial Sequence Synthetic polypeptide
MSIGLLCCAALSLLWAGPVNAGVTQTPKFQVLKTGQSMTLQCAQDMNHEYMSWYRQDPGMGLRLIHYSVAIQTTDRGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG